(19) United States
(12) Patent Application Publication
(10) Pub. No.: US 2022/0204971 A1
(43) Pub. Date: Jun. 30, 2022

(54) Compositions and Methods for Inhibiting Cancers and Viruses

(71) Applicants: Icahn School of Medicine at Mount Sinai, New York, NY (US); The Trustees of Princeton University, Princeton, NJ (US)

(72) Inventors: Maciej T. Nogalski, Princeton, NJ (US); Alexander Solovyov, New York, NY (US); Thomas Shenk, Princeton, NJ (US); Benjamin D. Greenbaum, New York, NY (US)

(73) Assignees: ICAHN SCHOOL OF MEDICINE AT MOUNT SINAI, New York, NY (US); The Trustees of Princeton University, Princeton, NJ (US)

(21) Appl. No.: 17/263,099

(22) PCT Filed: Jul. 25, 2019
(86) PCT No.: PCT/US19/43492

§ 371 Date: Jan. 25, 2021

Related U.S. Application Data
(60) Provisional application No. 62/703,378, filed on Jul. 25, 2018.
(60) Provisional application No. 62/748,771, filed on Oct. 22, 2018.

(51) Int. Cl. C12N 15/113

(52) U.S. Cl. CPC C12N 15/113; C12N 2310/3231; C12N 2310/113

(57) ABSTRACT

The present invention relates to compositions comprising isolated, single stranded RNA molecules and pharmaceutically acceptable carriers suitable for injection. The present invention relates to methods for stimulating an immune response and treating tumors. The present invention further relates to kits comprising a cancer vaccine and compositions of the present invention for use as an adjuvant to cancer vaccines.

This application is a U.S. national phase filing under 35 U.S.C. § 371 of PCT International Application No. PCT/US19/43492, filed Jul. 25, 2019, entitled, “Compositions and Methods for Inhibiting Cancers and Viruses,” which claims the benefit under 35 U.S.C. § 119(e) as a non-provisional of U.S. Provisional Patent Application Ser. Nos. 62/703,378, filed Jul. 25, 2018 and 62/748,771, filed Oct. 22, 2018, which are hereby incorporated by reference in their entirety.

This invention was made with government support under Grant No. AI112951 awarded by the National Institutes of Health. The government has certain rights in the invention.

### SEQUENCE LISTING

The instant application contains a Sequence Listing which has been submitted electronically in ASCII format and is hereby incorporated by reference in its entirety. Said ASCII copy, created on Aug. 29, 2019, is named MS-0008-01-US-NP_SL.txt and is 7,536 bytes in size.

### FIELD OF THE INVENTION

The present application relates to RNA containing compositions and methods of their use.

### BACKGROUND OF THE INVENTION

Repetitive sequences account for more than 50% of the human genome, while tandem satellite repeats account for 3% of the human genome (See, e.g., Levine et al., Bioessays 38, 508-513 (2016); Treangen, T. J. & Salzberg, S. L., Nat Rev Genet 13, 36-46 (2011)). Satellite DNA (satDNA) has been shown to form centromeric and pericentromeric loci and has been implicated in chromosome organization and segregation, kinetochore formation, and heterochromatin regulation. (See, e.g., Pezer Z. et al., Genome Dyn. 7, 153-169 (2012)). Recent developments in next generation sequencing (NSG) showed that these previously thought to be transcriptionally inert genomic sites could produce RNA transcripts and that those transcripts are actually accountable for the role of satDNA in chromosome and heterochromatin functions. (See, e.g., Chan, F. L. et al. PNAS, 109, 1979-1984 (2012); Bergmann, J. H. et al., J Cell Sci 125, 411-421 (2012).)

Human satellite repeat II (HSATII) and its mouse counterpart (GSAT) have are highly expressed in epithelial cancers and cancer cell lines but not in corresponding normal tissue. (See, e.g., Ting, D. T. et al., Science 331, 593-596, (2011); Leonova, K. I. et al., PNAS USA, 110, E89-98 (2013). While some satellite repeat transcription is stress-dependent or triggered during apoptotic differentiation or cell senescence programs, HSATII transcription has been shown to be refractory to these generalized environmental stressors and induced when cancer cells were grown in non-adherent conditions or as xenografts in mice. (See, e.g., De Cecco, M. et al., Aging Cell 12, 247-256 (2013). The sequence motifs of HSATII RNA mimic specifically some zoonotic viruses by containing CpG motifs within an AU-rich sequence context. These types of sequences are vastly underrepresented in the human genome, are avoided in viruses and immune-stimulatory in cells and are sensed by the antiviral protein ZAP if present in viral RNA¹⁷. (See, e.g., Tanne, A. et al., PNAS USA 15154-59 (2015); Takata, M. A. et al. Nature 24039 (2017)).

Human cytomegalovirus (HCMV), a β-herpesvirus, causes a chronic infection with lifelong latency in humans. (See, e.g., Tabata, T. et al., J Virol 89, 5134-47 (2015); Lanzieri, T. M., et al., Int J Infect Dis. 22, 44-48 (2014).) HCMV is a leading opportunistic pathogen in immunosuppressed individuals with infection capable to cause birth defects. HCMV strongly modulates cellular homeostasis for optimal viral replication and spread. It can be reactivated in the setting of reduced immunosurveillance²⁴, an immunological feature also observed in the emergence of cancers²⁵. (See, e.g., Gerna, G. et al., New Microbiol 35, 279-287 (2012); Tabata, T. et al., J Virol 89, 5134-47 (2105); Lanzieri, T. M., Int J Infect Dis, 22, 44-48 (2014).)

While prior work suggested that viral pathologies can be correlated with certain cancers, none demonstrated that HSATII expression plays a role in both diseases. The present invention overcomes these and other deficiencies in the prior art by showing that the HSATII induction seen in infected and cancer cells suggests possible convergence upon common HSATII-based regulatory mechanisms in these seemingly disparate diseases. In the case of HCMV, the present invention shows HSATII RNA is important for efficient viral protein expression and localization, viral replication and release of infectious particles. Moreover, the present invention shows HSATII function in several important cellular processes, including, for example, cellular motility. The present invention thus reveals a link between HSATII expression and virus-mediated pathobiology and shows that HSATII knockdown can reduce the accumulation of infectious virus.

### SUMMARY OF THE INVENTION

The present invention shows an acute induction of HSATII RNA in human cells that have been infected with two herpes viruses. It further shows that human cytomegalovirus (HCMV) IE1 and IE2 proteins cooperate to induce HSATII RNA affecting several aspects of the HCMV replication cycle and ultimately resulting in lower viral titers and altered infected-cell processes. The invention also demonstrates that post HCMV infection HSATII RNA synthesis is important for viral replication and viral pathogenesis. Furthermore, HSATII induction seen in infected and cancer cells shows common HSATII-based regulatory mechanisms that are targets for compositions directed to disease preventions and treatments.

One aspect of the present invention relates to a composition comprising an isolated, single stranded RNA molecule and a pharmaceutically acceptable carrier suitable for injection. The RNA molecules of the present invention may include additional nucleic acids at either end of the molecule that do not adversely affect the ability of the RNA to reduce the expression, function, or activity of HSATII. Conservative substitutions of nucleotides embedded within the RNA molecules of the present invention are also incorporated into the present invention by employing methods known to persons of skill in the art. “Conservative substitutions” are nucleotides that are functionally equivalent to a substituted nucleotide. As used herein, conservative substitutions do not disrupt the ability of the RNA molecule to inhibit or interfere with HSATII expression, function, or activity. An RNA molecule comprising one or more conservative substitutions is a conservative variant.

Another aspect of the present invention relates to a method of treating a subject for a viral infection, cancer, or a tumor. This method involves administering to a subject the composition of the present invention.

An embodiment of the present invention relates to an isolated RNA molecule and a pharmaceutically acceptable carrier suitable for injection, wherein the RNA molecule is an siRNA that reduces the expression, function, or activity of HSATII.

An embodiment of the present invention relates to an isolated RNA molecule and a pharmaceutically acceptable carrier suitable for injection, wherein the RNA molecule is a short hairpin RNA (shRNA) that reduces the expression, function, or activity of HSATII.

Another embodiment of the present invention relates to an isolated RNA molecule and a pharmaceutically acceptable carrier suitable for injection, wherein the RNA molecule is a locked nucleic acid (LNA) that reduces the expression, function, or activity of HSATII.

Another embodiment of the present invention relates to a kit that contains an isolated RNA molecule and a pharmaceutically acceptable carrier suitable for injection, wherein the RNA molecule is an siRNA that reduces the expression, function, or activity of HSATII.

Another embodiment of the present invention relates to a kit that contains an isolated RNA molecule and a pharmaceutically acceptable carrier suitable for injection, wherein the RNA molecule is an shRNA that reduces the expression, function, or activity of HSATII.

Another embodiment of the present invention relates to a kit that contains an isolated RNA molecule and a pharmaceutically acceptable carrier suitable for injection, wherein the RNA molecule is an LNA that reduces the expression, function, or activity of HSATII.

### BRIEF DESCRIPTION OF THE DRAWINGS

FIGS. 1a-f disclose the results of tests for HSATII expression in fibroblast and epithelial cells mock-infected or infected with HCMV, HSV1, adenovirus (Adv), influenza A, ZIKA, and hepatitis C viruses, including intracellular localization (FIGS. 1e, f). HFFs were infected with HCMV (3 TCID50/cell), HSV (3 TCID50/cell), or Ad5 (10 FFU/cell), and RNA samples were collected at 48, 9, or 24 hpi, respectively. RNA was isolated and analyzed using RNA-seq. a HSATII expression in terms of counts per million reads (CPM) was computed and normalized across samples. n=2. b HSATII chromosomal origin in infected cells or primary tumors was depicted based on the number of unique HSATII reads mapped to specific chromosomal loci. Data are presented as a percentage of total HSATII reads mean±SD. n=2. Open circles represent single data points. c HFFs were infected with HCMV (TB40/E-GFP) at 3 TCID50/cell and RNA samples were collected at the indicated times. HSATII-specific primers were used in RT-qPCR analysis. GAPDH was used as an internal control. Data are presented as a fold change mean±SD. n=3. d Fibroblasts were infected with HCMV (3 TCID₅₀/cell), HSV1 (3 TCID₅₀/cell), Ad5 (10 FFU/cell), FLU (3 TCID₅₀/cell), or ZIKV (10 PFU/cell), and Huh7 cells were infected with HCV (1 TCID₅₀/cell). RNA samples were collected at 9 hpi (HSV) or 24 hpi (all other viruses). HSATII-specific primers were used in RT-qPCR analysis. Viral infection was controlled by probing for a presence of viral transcripts: UL123 (HCMV), UL30 (HSV1), E2A (Ad5) or viral genomes: IAV and ZIKV. GAPDH was used as an internal control. Data are presented as a fold change mean±SD. n=3. Open circles represent single data points. e Mock- and HCMV (TB40/E-GFP)-infected HFFs at 3 TCID₅₀/cell were collected at 24 hpi and HSATII RNA was visualized by ISH assay. Nuclei were counterstained with hematoxylin and HSATII is shown as red dots. Scale bar: 50 μm. f HSATII signal from ISH staining was quantified based on the ratio of HSATII signal area to cell area using BDZ 6.0 software and is presented in box plots (a central line shows median and bounds of box the 25th and 75th percentiles) with 10-90 percentile whiskers. Dots represent outliers. n=3. ***P<0.001 by the unpaired, two-tailed t-test.

FIGS. 2a-d disclose HSATII induction levels in cells infected with active virus as compared to UV-irradiated virus. a HFFs were infected with untreated or UV-irradiated HCMV (TB40/E-GFP) at 3 TCID50/cell, RNA samples were collected at specified times. b HFFs were treated with CHX or DMSO, as a solvent control, 24 h before HCMV (TB40/E-GFP) infection at 1 TCID50/cell. RNA samples were collected at 24 hpi. c HFFs were infected with HCMV (TB40/E-GFP) at 1 TCID50/cell for 2 h and then media was changed for one containing GCV or DMSO as a solvent control. RNA samples were collected at 24 and 48 hpi. d Tetracycline-inducible TE1 and/or IE2 MRC-5 and ARPE-19 cells were treated with doxycycline. RNA samples were collected at indicated times. a-d RT-qPCR was performed using HSATII-specific primers. GAPDH was used as an internal control. n=3. Data are presented as a fold change mean±SD. a-c ***P<0.001, ****P<0.0001 by the unpaired, two-tailed t-test with (b, c) or without (a) Welch's correction. ns—not significant. Open circles represent single data points.

FIGS. 3a-d disclose the results of RNA sequence analysis directed to detecting HSATII transcripts in HCMV-infected cells as compared with NT-LNA transfected cells. RNA samples were collected at 24 hpi from HFFs transfected with NT-LNA or HSATII-LNAs 24 h before HCMV (TB40/E-GFP) infection at 1 TCID₅₀/cell. a RT-qPCR was performed using HSATII-specific primers. GAPDH was used as an internal control. Data are presented as a fold change mean±SD. n=3. ***P<0.001, ****P<0.0001 by the unpaired, two-tailed t-test with Welch's correction. Open circles represent single data points. b RNA-seq analysis performed. Only unique HSATII reads were used to calculate its expression. HSATII expression in terms of CPM was computed and normalized across samples. n=2. Open circles represent single data points. c Media samples were collected at indicated times from HFFs transfected with NT-LNA or HSATII-LNAs 24 h before HCMV (TB40/E-GFP) infection at 1 TCID₅₀/cell. TCID₅₀ ml⁻¹ values were determined. n=3. *P<0.05, **P<0.01 by the unpaired, two-tailed t-test. d Media samples were collected at 96 hpi from HFFs transfected with pcDNA or pcDNA-HSATII 48 h before HCMV (TB40/E-GFP) infection at 1 TCID₅₀/cell. % of infected cells was calculated based on a number of IE1-positive cells in a reporter plate. Data are presented as a fold change mean±SD. n=3. *P<0.05 by the unpaired, two-tailed t-test with Welch's correction. Open circles represent single data points. Inside panel: RNA samples were collected from pcDNA or pcDNA-HSATII-transfected and HCMV-infected HFFs. HSATII-pcDNA primer set was used in RT-PCR analysis. B2M was used as an internal control. NTC non-template control sample.

FIGS. 4a-e disclose assays of the expression and localization of various proteins (IE1, IE2, pUL26, pUL44, pUL69, and pUL99) in infected cells in the presence of HSATII-LNAs in infected cells. a Protein samples were collected at indicated times from HFFs transfected with NT-LNA or HSATII-LNAs 24 h before HCMV (TB40/E-GFP) infection at 1 TCID50/cell. Protein levels were analyzed by the western blot technique using antibodies specific to IE1, IE2, UL26, pUL44, pUL69, and pp28. Actin was used as a loading control. b HFFs were transfected with NT-LNA or HSATII-LNAs 24 h before HCMV (TB40/E) infection at 1 TCID50/cell. At 72 hpi, cells were fixed and stained for IE1, ppUL44, p28 or gB and nuclei were counterstained with the Hoechst stain. Scale bar: 15 μm. c Total DNA was collected at indicated times from HFFs transfected with NT-LNA or HSATII-LNAs 24 h before HCMV (TB40/E-GFP) infection at 1 TCID50/cell. vDNA and cellular DNA copy numbers were determined. Data are presented as a fold change mean±SD of the relative vDNA to cellular DNA ratio. n=3. *P<0.05 by the unpaired, two-tailed t-test. Open circles represent single data points. d Intracellular and extracellular virions were collected at indicated times from HFFs transfected with NT-LNA or HSATII-LNAs 24 h before HCMV (TB40/E-GFP) infection at 1 TCID50/cell. TCID50 ml-1 values were determined. Data are presented as a mean±SD. n=3. **P<0.01, ***P<0.001 by the unpaired, two-tailed t-test. Open circles represent single data points. e Particle-to-TCID50 ratios were calculated based on the TCID50 assay and vDNA copy numbers generated from media samples collected at 96 hpi from HFFs transfected with NT-LNA or HSATII-LNAs 24 h before HCMV (TB40/E-GFP) infection at 1 TCID50/cell. Data are presented as a particle-to-TCID50 ratio mean±SD. n=5. ***P<0.001 by the unpaired, two-tailed t-test. Open circles represent single data points.

FIGS. 5a-d disclose analyses of differentially regulated RNAs induced by HSATII RNA in mock and HCMV infected cells. a HSATII regulates expression of cellular genes. RNA samples were collected at 24 hpi from HFFs transfected with NT-LNA or HSATII-LNAs 24 h before mock or HCMV (TB40/E-GFP) infection at 1 TCID₅₀/cell. RNA was isolated and analyzed using RNA-seq. GSEA was performed on the list of cellular genes differentially expressed in HCMV-infected, NT-LNA—versus HSATII-LNA-transfected HFFs. The matrix shows genes overlapping with specific gene set names (numbered) categorized based on increasing P-value and FDR q-value. GSEA-identified enriched gene sets: 1—HALLMARK EPITHELIAL MESENCHYMAL TRANSITION; 2—GHANDHI BYSTANDER IRRADIATION UP; 3—SATO SILENCED BY DEACETYLATION IN PANCREATIC CANCER; 4—GO CELLULAR RESPONSE TO ORGANIC SUBSTANCE; 5—NABA MATRISOME; 6—HAN SATB1 TARGETS DN; 7—DELYS THYROID CANCER UP; 8—ONDER CDH1 TARGETS 2 DN; 9—GHANDHI DIRECT IRRADIATION UP; 10—WANG SMARCE1 TARGETS DN. b, c HSATII regulates motility of epithelial cells. ARPE-19 cells were transfected with NT-LNA or HSATII-LNAs 24 h before mock or HCMV (TB40-epi) infection at 1 TCID₅₀/cell. After 2 hpi, wound was created and its closure was monitored. The graph shows a wound closure at 44 hpi. Data from biological replicates are presented as a percent of remaining wound width mean±SD. n=4. **P<0.01, ***P<0.001 by the unpaired, two-tailed t-test. Open circles represent single data points. c ARPE-19 cells were transfected with NT-LNA or HSATII-LNAs 24 h before mock or HCMV (TB40-epi) infection at 1 TCID₅₀/cell. After 6 hpi, cells were transferred onto transwell inserts. Migrated cells were washed, fixed, and nuclei stained. The graph presents a fold change mean±SD based on a number of cells migrated through a transwell per FOV. Data from biological replicates are presented as a fold change mean±SD. n=5. **P<0.01, ***P<0.001 by the unpaired, two-tailed t-test with Welch's correction. Open circles represent single data points. d HSATII is markedly elevated in HCMV colitis. Paraffin-embedded sections of normal epithelium, low HCMV antigen-positive, or high HCMV antigen-positive CMV colitis sections were processed. HSATII RNA was visualized by ISH assay using HSATII-specific probe. An intensely brown stain characterizes CMV antigen-positive cells. Nuclei were counterstained with hematoxylin (purple stain) and HSATII is shown as red stain. Scale bar: 100 μm.

FIGS. 6a-b disclose total RNA-seq showing both coding and non-coding transcriptomes of acute HCMV infection in human foreskin fibroblasts showing infected (FIG. 6a) and mock-infected (FIG. 6b) cells. HFFs were infected with HCMV (AD169) at 3 TCID₅₀/cell and RNA samples were collected at 48 hpi. RNA was isolated and analyzed using RNA-seq. Differential expression of transcripts in infected cells was computed based on their expression in mock-infected cells. The q-value <0.05 and a fold change ±2 were used as significance thresholds. a The pie chart depicts differentially regulated coding, non-coding and repeat element transcripts in HCMV-infected fibroblasts at 48 hpi. The bar graphs represent a percent of upregulated (red bars) and downregulated (green bars) transcripts in each class. b Several classes of repeat elements are differentially regulated during HCMV infection. The graph presents cumulative RNA-seq data analysis from cells infected with AD169, TB40, FIX or TB40e strains of HCMV at 3 TCID₅₀/cell. Depicted are only repeat elements that are differentially expressed in each infection. The q-value computed for an individual repeat element in the RNA-seq analysis of each infection experiment.

FIG. 7 discloses HSATII expression levels over time in HCMV-infected ARPE-19 epithelial cells. ARPE-19 cells were infected with HCMV (TB40-epi) at 3 TCID₅₀/cell and RNA samples were collected at the indicated time points. HSATII-specific primers were used in RT-qPCR analysis. GAPDH was used as an internal control. Data were averaged from at least three experiments and are presented as a fold change mean (SD).

FIGS. 8a-b disclose the percentage of cells infected with HCMV, HSV1, Ad5, IAV, or ZIKA. HFFs were infected with HCMV (TB40/E-GFP; 3 TCID₅₀/cell), HSV1 (3 TCID₅₀/cell), Ad5 (10 FFU/cell), IAV (3 TCID₅₀/cell) or ZIKV (10 PFU/cell) and fixed at 24 hpi (HCMV and Ad5) or 12 hpi (HSV1 and IAV). Cells were stained for IE1 (HCMV), ICP4 (HSV1), DBP (Ad5), NP (IAV) or the flavivirus antigen (ZIKV) and nuclei were counterstained with the Hoechst stain. Cells were visualized (a) and % of viral antigen-positive cells was calculated (b) using Operetta high-content imaging and analysis system.

FIG. 9 discloses the detection of HSATII transcripts in cells infected with HCMV, mock infected cell, and cells in the presence or absence of reverse transcriptase. RNA samples were collected at 24 hpi from mock- or HCMV (TB40/E-GFP)-infected cells at 1 TCID₅₀/cell. RNA underwent RT reaction with or without reverse transcriptase and HSATII-specific primers were used to quantify HSATII expression by qPCR. GAPDH was used as an internal control. Data were averaged from at least three experiments and are presented as a mean (SD).

FIG. 10 discloses that HCMV mRNA, UL123, HSATII RNA cells from infected cells is not retained on an oligo-dT matrix or efficiently amplified from oligo dT-based cDNA. HFFs were infected with HCMV (TB40/E-GFP) at 1 TCID₅₀/cell and total RNA was collected at 24 hpi. Total RNA with or without enriching for polyA-tailed transcripts underwent RT reaction using random hexamers or oligo-dT. HSATII- and UL123-specific primers were used in RT-qPCR analysis. GAPDH was used as an internal control. Data were averaged from at least three experiments and are presented as a fold change mean (SD).

FIG. 11 discloses virion protein levels in cells infected in cells infected with active virus as compared to UV-irradiated virus. HFFs were infected with untreated or UV-irradiated HCMV (TB40/E-GFP; 3 TCID₅₀/cell) and protein samples were collected at specified times. Protein levels were analyzed by western blotting using anti-pp71 antibody. Actin was used as a loading control.

FIG. 12 discloses the accumulation of UL99 gene RNA in the presence of DMSO or GCV in infected cells. HFFs were infected with HCMV (TB40/E-GFP; 1 TCID₅₀/cell) for 2 h and then media was changed for one containing GCV or DMSO as a solvent control. RNA samples were collected at 48 hpi. RT-qPCR was performed using UL99-specific primers. GAPDH was used as an internal control. Data were averaged from at least three experiments and are presented as a fold change mean (SD).

FIGS. 13a-b disclose protein expression in infected MRC-5, ARPE-19, Fibroblast, or Epithelial cells. a Tetracycline-inducible MRC-5 and ARPE-19 cells were treated with doxycycline for 24, 48 or 72 h or b HFF and ARPE19 cells were infected with HCMV (TB40/E-GFP and TB40-epi, respectively) at 1 TCID₅₀/cell. Protein samples were collected at indicated times. Protein levels were analyzed by western blotting using with anti-GFP, anti-IE1 or anti-IE2 antibodies. Actin was used as a loading control.

FIG. 14 discloses the effect on cell viability of locked nucleic acids (LNAs) that target HSATII transcripts. HFFs were transfected with increasing concentrations of NT-LNA or HSATII-LNAs. Cells were mock- or HCMV (TB40/E-GFP)-infected at 1 TCID₅₀/cell and cell viability was assessed at 48 h post LNA transfection (hpt) and 24 hpi or 120 hpt and 96 hpi. Data is presented as % viable cells, were averaged from at least three experiments and are presented as mean (SD).

FIGS. 15a-c disclose the expression levels of certain RNA transcripts in HCMV infected and mock infected cells in the presence of certain LNAs. a HSATII-specific LNAs alter expression of protein-coding cellular RNAs in HCMV-infected fibroblasts. b HSATII-specific LNAs are highly specific for HSATII among non-coding cellular repeat RNAs. c HSATII-specific LNAs do not alter expression of HCMV transcripts at 24 hpi. (a,b,c) RNA samples were collected at 24 hpi from HFFs transfected with NT-LNA or HSATII-LNAs 24 h before mock or HCMV (TB40/E-GFP) infection at 1 TCID₅₀/cell. RNA was isolated and analyzed using RNA-seq. Differential expression of transcripts in HSATII-deficient cells was computed based on their expression in NT-LNA-transfected cells. Volcano plots were generated based on differential fold change expression of transcripts and the computed q-values between mock-infected, NT-LNA- and HSATII-LNA-transfected cells (blue dots) or between HCMV-infected, NT-LNA- and HSATII-LNA-transfected cells (orange dots).

FIG. 16 discloses the effect of certain HSATII-LNAs on HCMV titer. RNA samples were collected at 96 hpi from HFFs transfected with NT-LNA or HSATII-LNAs 24 h before HCMV (TB40/E-GFP) infection at 1 TCID₅₀/cell. RT-qPCR was performed using UL123, UL122, UL37xl, UL26, UL54, UL69, UL82, UL99, RNA4.9 and RNA5.0 specific primers. GAPDH was used as an internal control. Data were averaged from at least three experiments and are presented as a fold change mean (SD).

FIGS. 17a-b discloses HCMV genome and HCMV coding RNAs are characterized by CpG motif overrepresentation, but not in a background of AU-rich sequences. Histograms of forces (strength of statistical bias) on CpG for (a) HCMV genome and (b) HCMV coding RNAs compared to the frequency of AU-dinucleotides.

FIG. 18 discloses HSATII-LNAs do not affect differential expression of HCMV transcripts. Differential expression of HCMV transcripts at 24 hpi between NT-LNA- and HSATII-LNA-treated fibroblasts were plotted against the alignment score generated based on sequence similarity of the corresponding HCMV transcript sequence and HSATII-LNAs.

FIG. 19 discloses HSATII RNA affects cellular localization of HCMV late proteins. HFFs were transfected with NT-LNA or HSATII-LNAs 24 h before HCMV (TB40/E-GFP) infection at 1 TCID50/cell. At 72 hpi, cells were fixed and stained for IE1, pp28 or gB and nuclei were counterstained with the Hoechst stain. Cells were visualized using Nikon Ti-E with spinning disc.

FIG. 20 discloses the effect of DNAse on viral and cellular DNA levels. DNA samples were collected from HCMV (TB40/E-GFP)-infected HFFs at 1 TCID₅₀/cell and treated or not treated with DNase. qPCR was performed using gUL123, gUL44 and gGAPDH specific primers. Ct values were averaged from at least two experiments and are presented as a mean (SD).

FIG. 21 discloses assays of HSATII regulation on several cellular processes. RNA was collected at 24 hpi from HFFs transfected with NT-LNA or HSATII-LNAs 24 h before mock or HCMV (TB40/E-GFP) infection at 1 TCID₅₀/cell. RNA was isolated and analyzed using RNA-seq. Differential expression of transcripts in HSATII-deficient cells was computed based on their expression in NT-LNA-transfected cells. A graphical representation of results of Core Analysis in IPA performed on a group of genes with expression significantly changed between HCMV-infected, NT-LNA- and HSATII-LNA-transfected HFFs. Genes were organized based on statistically enriched GO groups.

FIGS. 22a-b discloses representative colitis samples stained for a presence of CMV antigen. Paraffin-embedded sections of low (a: panel 1; b: panels 1 and 2) and high (a: panel 2; b: panels 3 and 4) grade CMV colitis (a) commonly IHC stained for CMV antigens and (b) IHC stained for HCMV IE2 (brown stain). Nuclei were counterstained with hematoxylin (purple stain).

FIGS. 23a-g discloses development of RT-qPCR-based assay for a quantitative evaluation of HSATII expression. a-e—standard curves demonstrating a linear increase of HSATII amplicons with an increasing concentration of cDNA sample. f A standard curve demonstrating a linear increase of GAPDH amplicon with an increasing concentration of cDNA sample. g A graphical depiction of HSATII chromosomal loci showing binding locations of HSATII-specific primers. The orange color marks HSATII consensus sequence repeat.

FIG. 24 discloses the effects of four different LNAs on HSATII RNA levels. RNA samples were collected at 24 hpi from HFFs transfected with NT-LNA or different HSATII-LNAs 24 h before HCMV (TB40/E-GFP) infection at 1 TCID₅₀/cell. RT-qPCR was performed using HSATII-specific primers. GAPDH was used as an internal control. Data were averaged from at least three independent experiments and are presented as a fold change mean (SD). Unpaired, two-tailed t-test was used to measure significance. The asterisk represents p<0.05.

FIG. 25 discloses the effect of four different HSAT-II LNAs on production of HCMV infectious particles from human foreskin fibroblasts (HFF). Media samples were collected at 96 hpi from HFFs transfected with NT-LNA or different HSATII-LNAs 24 h before HCMV (TB40/EGFP) infection at 1 TCID₅₀/cell. TCID₅₀/ml values were determined. Data were averaged from at least three independent experiments and are presented as a fold change mean (SD). Unpaired, two-tailed t-test was used to measure significance. One, two or three asterisks represent p<0.05, p<0.01, and p<0.001, respectively.

FIG. 26 discloses the effect of HSATII knockdown using four different HSAT-II LNAs on the production of HCMV infectious particles from human retinal pigment cells. Media samples were collected at 96 hpi from ARPE-19 cells transfected with NT-LNA or different HSATII-LNAs 24 h before HCMV (TB40-epi) infection at 1 TCID50/cell. PFU/ml values were determined. Data were averaged from at least three independent experiments and are presented as a mean (SD). Unpaired, two-tailed t-test was used to measure significance. One, two, three or four asterisks represent p<0.05, p<0.01, p<0.001 or p<0.0001, respectively.

### DETAILED DESCRIPTION OF THE INVENTION

The invention described herein relates to RNA-containing compositions and methods of their use.

In a first aspect, the present invention relates to a composition comprising an isolated, single stranded RNA molecule having homology to HSATII. In another aspect, the present invention relates to a small interfering RNA molecule (siRNA) having homology to HSATII. In yet another aspect, the present invention relates to locked nucleic acids (LNAs) having homology to HSATII.

In one embodiment, the composition comprises a pharmaceutical composition containing an isolated RNA molecule in the form of a vaccine or a pharmaceutical composition in the form of an adjuvant to a vaccine.

In one embodiment, the RNA molecule in the composition of the present invention is an isolated RNA molecule. The term “isolated RNA molecule” includes RNA molecules that are separated from other nucleic acid molecules that are present in the natural source of the RNA. An “isolated” nucleic acid molecule is free of sequences that naturally flank the nucleic acid (i.e., sequences located at the 5′ and 3′ ends of the nucleic acid molecule). An “isolated” nucleic acid molecule is substantially free of other cellular material, or culture medium, when produced by recombinant techniques, or substantially free of chemical precursors or other chemicals when chemically synthesized.

Suitable RNA molecules in the composition of the present invention include, without limitation, an RNA molecule having the nucleotide sequence of HSATII or that is complementary to HSATII or a fragment thereof. Such RNA molecules can be isolated using standard molecular biology techniques and the sequence information provided herein. In one embodiment, using all or a portion of the nucleic acid sequence of HSATII as a hybridization probe, RNA molecules can be isolated using standard hybridization and cloning techniques.

Moreover, an RNA molecule in the composition of the present invention can be isolated by the polymerase chain reaction (PCR) using synthetic oligonucleotide primers. In one embodiment, the primers are designed based on the sequence (or a portion thereof) of HSATII.

The RNA molecules in the composition of the present invention has an immunostimulating effect on cells, including tumor cells. As used herein, the term “immunostimulating effect” or “stimulating an immune response” includes eliciting an immune response, e.g., inducing or increasing T cell-mediated and/or B cell-mediated immune responses that are influenced by modulation of T cell costimulation. Exemplary immune responses include B cell responses (e.g., antibody production), T cell responses (e.g., cytokine production, and cellular cytotoxicity), and activation of cytokine responsive cells, e.g., macrophages. Eliciting an immune response includes an increase in any one or more immune responses. It will be understood that upmodulation of one type of immune response may lead to a corresponding downmodulation in another type of immune response. For example, upmodulation of the production of certain cytokines (e.g., IL-10) can lead to downmodulation of cellular immune responses. The RNA molecule elicits an immuno-stimulating effect on immune cells. As used herein, the term “immune cell” includes cells that are of hematopoietic origin and that play a role in the immune response. Immune cells include lymphocytes, such as B cells and T cells; natural killer cells; and myeloid cells, such as monocytes, macrophages, eosinophils, mast cells, basophils, and granulocytes. The term “T cell” includes CD4+ T cells and CD8+ T cells. The term T cell also includes both T helper 1 type T cells and T helper 2 type T cells.

In embodiments of the present invention, the RNA molecule is incorporated into pharmaceutical compositions suitable for administration (e.g., by injection). Such compositions typically comprise the RNA molecule and a carrier, e.g., a pharmaceutically acceptable carrier. The pharmaceutically acceptable carrier suitable for injection is, according to one embodiment, a carrier for the RNA molecule. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. Supplementary active compounds can also be incorporated into the compositions.

The pharmaceutically acceptable carrier may be a stabilizer, an emulsion, liposome, microsphere, immune stimulating complex, nanospheres, montanide, squalene, cyclic dinucleotides, complementary immune modulators, or any combination thereof. The carrier should be suitable for the desired mode of delivery of the composition (i.e., suitable for injection). Exemplary modes of delivery include, without limitation, intravenous injection, intra-arterial injection, intramuscular injection, intracavitary injection, subcutaneously, intradermally, transcutaneously, intrapleurally, intraperitoneally, intraventricularly, intra-articularly, intraocularly, intratumorally, or intraspinally.

Pharmaceutical compositions of the invention are formulated to be compatible with their intended route of administration. Solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates, or phosphates; and agents for the adjustment of tonicity such as sodium chloride or dextrose. pH can be adjusted with acids or bases, such as hydrochloric acid or sodium hydroxide. The parenteral preparation can be enclosed in ampoules, disposable syringes, or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include, for example, physiological saline, bacteriostatic water, or phosphate buffered saline (PBS). The composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. It may be preferable to include isotonic agents, for example, sugars, polyalcohols such as manitol, sorbitol, and sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (i.e., RNA molecule) in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

It is especially advantageous to formulate parenteral compositions in dosage unit form for ease of administration and uniformity of dosage. Dosage unit form as used herein refers to physically discrete units suited as unitary dosages for the subject to be treated; each unit containing a predetermined quantity of active compound (i.e., RNA molecule) calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

Toxicity and therapeutic efficacy of such compounds can be determined by standard pharmaceutical procedures in cell cultures or experimental animals. The data obtained from the cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration utilized. For any compound used in the methods of the invention (described infra), the therapeutically effective dose can be estimated initially from cell culture assays. A dose may be formulated in animal models to achieve a circulating plasma concentration range that includes the IC50 (i.e., the concentration of the test compound which achieves a half-maximal activity) as determined in cell culture. Such information can be used to more accurately determine useful doses in humans. Levels in plasma may be measured, for example, by high performance liquid chromatography.

As defined herein, a therapeutically effective amount of an RNA molecule (i.e., an effective dosage) ranges from about 0.001 to 30 mg/kg body weight, or about 0.01 to 25 mg/kg body weight, or about 0.1 to 20 mg/kg body weight, or about 1 to 10 mg/kg, 2 to 9 mg/kg, 3 to 8 mg/kg, 4 to 7 mg/kg, or 5 to 6 mg/kg body weight. The skilled artisan will appreciate that certain factors may influence the dosage required to effectively treat a subject, including but not limited to, the severity of the disease or disorder, previous treatments, the general health and/or age of the subject, and other diseases present. Moreover, treatment of a subject with a therapeutically effective amount of an agent can include a single treatment or, preferably, can include a series of treatments.

In one embodiment, a subject is treated with the composition of the present invention in the range of between about 0.1 to 20 mg/kg body weight, one time per week for between about 1 to 10 weeks, preferably between 2 to 8 weeks, more preferably between about 3 to 7 weeks, and even more preferably for about 4, 5, or 6 weeks. It will also be appreciated that the effective dosage of composition used for treatment may increase or decrease over the course of a particular treatment. Changes in dosage may result and become apparent from the results of diagnostic assays.

In one embodiment, nucleic acid molecules can be inserted into vectors and used as gene therapy vectors. Gene therapy vectors can be delivered to a subject by, for example, intravenous injection, local administration (U.S. Pat. No. 5,328,470, which is hereby incorporated by reference in its entirety) or by stereotactic injection (Chen et al., “Regression of Experimental Gliomas by Adenovirus-Mediated Gene Transfer In Vivo,” Proc. Natl. Acad. Sci. USA 91:3054-3057 (1994), which is hereby incorporated by reference in its entirety). The pharmaceutical preparation of the gene therapy vector can include the gene therapy vector in an acceptable diluent or can comprise a slow release matrix in which the gene delivery vehicle is imbedded. Alternatively, where the complete gene delivery vector can be produced intact from recombinant cells, e.g., retroviral vectors, the pharmaceutical preparation can include one or more cells which produce the gene delivery system. The pharmaceutical compositions can be included in a container, pack, or dispenser together with instructions for administration.

The composition of the present invention can also include an effective amount of an additional adjuvant or mitogen.

Suitable additional adjuvants include, without limitation, Freund's complete or incomplete, mineral gels such as aluminum hydroxide, surface active substances such as lysolecithin, pluronic polyols, polyanions, peptides, oil emulsions, dinitrophenol, Bacille Calmette-Guerin, Carynebacterium parvum, non-toxic Cholera toxin, N-acetyl-muramyl-L-threonyl-D-isoglutamine (thr-MDP), N-acetyl-nor-muramyl-L-alanyl-D-isoglutamine (CGP 11637, referred to as nor-MDP), N-acetylmuramyl-L-alanyl-D-isoglutaminyl-L-alanme-2-(r-2′-dipalmitoyl-s-n-glycero-3-hydroxyphosphoryloxy)-ethylamine (CGP 19835 A, referred to as MTP-PE), and RIBI, which contains three components extracted from bacteria, monophosphoryl lipid A, trehalose dimycolate, and cell wall skeleton (MPL+TDM+CWS) in a 2% squalene/TWEEN® 80 emulsion.

As used herein, “mitogen” refers to any agent that stimulates lymphocytes to proliferate independently of an antigen. The mitogen, in combination with the RNA molecule in the composition of the present invention helps to promote an immuno-stimulating effect on tumor cells. Exemplary mitogen include, without limitation, CpG oligodeoxynucleotides that stimulate immune activation as described in U.S. Pat. Nos. 6,194,388; 6,207,646; 6,214,806; 6,218,371; 6,239,116; 6,339,068; 6,406,705; and 6,429,199, each of which is hereby incorporated by reference in its entirety. Any suitable dosage of mitogen can be used to promote an immuno-stimulating effect on tumor cells. For example, a suitable dosage of mitogen comprises about 50 ng up to about 100 jug per ml, about 100 ng up to about 25 lag per ml, or about 500 ng up to about 5 μg per ml.

The composition may also include an antigen or an antigen-encoding RNA molecule. As used herein, “antigen” refers to any agent that induces an immune response, i.e., a protective immune response, against the antigen, and thereby affords protection against a pathogen or disease (e.g., cancer). The antigen can take any suitable form including, without limitation, whole virus or bacteria; virus-like particle; anti-idiotype antibody; bacterial, viral, or parasite subunit vaccine or recombinant vaccine; and bacterial outer membrane (“OM”) bleb formations containing one or more of bacterial OM proteins.

The antigen can be present in the compositions in any suitable amount that is sufficient to generate an immunologically desired response. The amount of antigen or antigen-encoding RNA molecule to be included in the composition will depend on the immunogenicity of the antigen itself and the efficacy of any adjuvants co-administered therewith. In general, an immunologically or prophylactically effective dose comprises about 1 μg to about 1,000 μg of the antigen, about 5 μg to about 500 μg, or about 10 μg to about 200 μg.

According to another embodiment, the composition (i.e., a first pharmaceutical composition) may further include a cancer vaccine (i.e., as a second pharmaceutical composition) that includes an antigen or a nucleic acid molecule encoding the antigen, and a pharmaceutically suitable carrier. According to this embodiment, the first pharmaceutical composition is intended to be co-administered with the second pharmaceutical composition for purposes of enhancing the efficacy of the vaccine. The first pharmaceutical composition is formulated for and/or administered in a manner that achieves an immuno-stimulating effect on tumor cells.

Cancer vaccines are known, and include, for example, sipuleucel-T, which is approved for use in some men with metastatic prostate cancer. This vaccine is designed to stimulate an immune response to prostatic acid phosphatase (“PAP”), an antigen that is found on most prostate cancer cells. Sipuleucel-T′ is customized to each patient. The vaccine is created by isolating immune system cells called antigen-presenting cells (“APCs”) from a patient's blood through a procedure called leukapheresis. The APCs are sent to Dendreon, where they are cultured with a protein called PAP-GM-C SF. This protein consists of PAP linked to another protein called granulocyte-macrophage colony-stimulating factor (GM-CS F). The latter protein stimulates the immune system and enhances antigen presentation. APC cells cultured with PAP-GM-CSF constitute the active component of sipuleucel-T. Each patient's cells are returned to the patient's treating physician and infused into the patient. Patients receive three treatments, usually 2 weeks apart, with each. round of treatment requiring the same manufacturing process. Although the precise mechanism of action of stpuleucel-T is not known, it appears that the APCs that have taken up PAP-GM-CSF stimulate T cells of the immune system to kill tumor cells that express PAP.

Vaccines to prevent HIPV infection and to treat several types of cancer are being studied in clinical trials. Active clinical trials of cancer treatment vaccines include vaccines for bladder cancer, brain tumors, breast cancer, cervical cancer, Hodgkin lymphoma, kidney cancer, leukemia, lung cancer, melanoma, multiple myeloma, non-Hodgkin lymphoma, pancreatic cancer, prostate cancer, and solid tumors. Active clinical trials of cancer preventive vaccines include those for cervical cancer and solid tumors. Cancer vaccines approved from these and other trials may be suitable cancer vaccines for use in combination with the composition of the present invention.

Another aspect of the present invention relates to a kit comprising a cancer vaccine and the composition of the present invention, as well as instructions and a suitable delivery device, which can optionally be pre-filled with the vaccine formulation (i.e., the composition of the present invention and the cancer vaccine). An exemplary delivery device includes, without limitation, a syringe comprising an injectable dose.

A further aspect of the present invention relates to a method of treating a subject for a tumor. This method involves administering to a subject the composition of the present invention under conditions effective to treat the subject for the tumor.

In one embodiment of this and other methods described herein, the subject is a mammal including, without limitation, humans, non-human primates, dogs, cats, rodents, horses, cattle, sheep, and pigs. Both juvenile and adult mammals can be treated. The subject to be treated in accordance with the present invention can be a healthy subject, a subject with a tumor, a subject with cancer, a subject being treated for cancer, a subject in cancer remission, or a subject that has an immune deficiency or is immunosuppressed. Although otherwise healthy, the elderly and the very young may have a less effective (or less developed) immune system and they may benefit greatly from the enhanced immune response.

Tumors include, without limitation, sarcoma, melanoma, lymphoma, leukemia, neuroblastoma, or carcinoma cell tumors.

In carrying out this and the other methods described herein, administering may be carried out as described supra, including, for example, intratumorally or systemically using a pharmaceutical composition as described supra, and amounts, dosages, and administration frequencies described supra.

A further aspect of the present invention relates to a method of stimulating an immune response against cancer in a cell or tissue. This method involves providing the composition of the present invention and contacting a cell or tissue with the composition under conditions effective to stimulate an immune response against cancer in the cell or tissue.

Cancers suitable for treatment in carrying out this aspect of the present invention include, for example and without limitation, those that are incident to pathogen infection, e.g., cervical cancer, vaginal cancer, vulvar cancer, oropharyngeal cancers, anal cancer, penile cancer, and squamous cell carcinoma of the skin caused by papillomavirus infection (D'Souza et al, “Case-Control Study of Human Papillomavirus and Oropharyngeal Cancer,” NEJM 356(19):1944-1956 (2007); Harper et al., “Sustained Immunogenicity and High Efficacy Against HPV 16/18 Related Cervical Neoplasia: Long-term Follow up Through 6.4 Years in Women Vaccinated with Cervarix (GSK's HPV-16/18 ASO4 candidate vaccine),” Gynecol. Oncol. 109:158-159 (2008), each of which is hereby incorporated by reference in its entirety) and liver cancer caused by Hepatitis B virus infection (Chang et al., “Decreased Incidence of Hepatocellular Carcinoma in Hepatitis B Vaccines: A 20-Year Follow-up Study,” J. Natl. Cancer Inst. 101:1348-1355 (2009), which is hereby incorporated by reference in its entirety) and Hepatitis C virus infection, Burkitt lymphoma, non-Hodgkin lymphoma, Hodgkin lymphoma, nasopharyngeal carcinoma caused by the Epstein-Barr virus, Kaposi sarcoma caused by the Kaposi sarcoma-associated herpesvirus, adult T-cell leukemia/lymphoma, caused by the human T-cell lymphotropic virus type 1, stomach cancer, mucosa-associated lymphoid tissue lymphoma caused by the bacterium Helicobacter pylori, bladder cancer caused by the parasite Schistosoma hematobium, and cholangiocarcinoma caused by the parasite Opisthorchis viverrini. An enhanced immune response achieved by the methods of treatment and compositions of the present invention may enhance the preventative efficacy of such vaccines for the prevention of cancers.

In one embodiment, this and other methods of the present invention are carried out to treat cancers that have already developed in a subject. Thus, the methods and compositions of the present invention are intended to delay or stop cancer cell growth: to cause tumor shrinkage; to prevent cancer from coming back: or to eliminate cancer cells that have not been killed by other forms of treatment.

According to one embodiment, a composition to be administered includes the antigen that is intended to generate the desired immune response as well as the RNA molecule. Thus, the antigen and the RNA molecule are co-administered simultaneously. The composition may be administered as a vaccine in a single dose or in multiple doses, which can be the same or different.

This embodiment may optionally include further administration of a composition of the present invention that includes the RNA molecule but not the antigen. This composition can be administered once or twice daily within several days preceding vaccine administration and for a period of time following vaccine administration. By way of example, post-vaccine administration can be carried out for up to about six weeks following each vaccine administration, preferably at least about two to three weeks, or at least about 3 to 10 days following each vaccine administration.

According to another embodiment, a vaccine composition to be administered includes the antigen that is intended to generate the desired immune response but not the RNA molecule. However, the RNA molecule can be co-administered at about the same time. For instance, the dosage of the vaccine can be administered interperitoneally or intranasally, and a dosage of the RNA molecule can be administered orally at about the same time (same day). The dosage containing the RNA molecule can also be once or twice administered daily for up to about six weeks following the vaccine administration.

In carrying out this method of the present invention, contacting the cell or tissue with the composition may be carried out in vitro or in vivo.

According to another aspect of the present invention, the RNA-containing composition has an immune-stimulating effect that primes (e.g., stimulates, induces, enhances, alters, or modulates) the anti-pathogen response of a subject's innate immune system in non-tumor cells. Such a response may find use, e.g., as an adjuvant to a vaccine, a vaccine supplement, or under conditions where such an immune-stimulating effect is desirable.

The present invention may be further illustrated by reference to the following examples, which should not be construed as limiting.

### EXAMPLES

### Example 1—HSATII Expression in HCMV Infected Cells

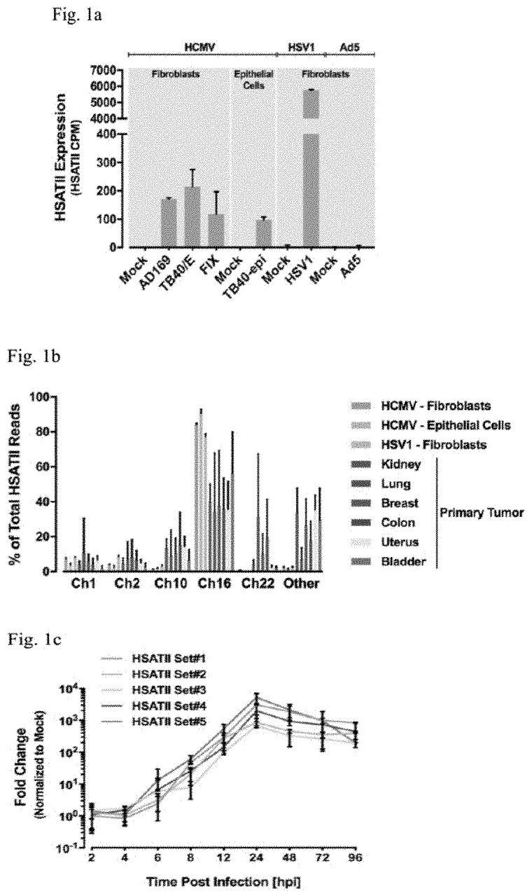

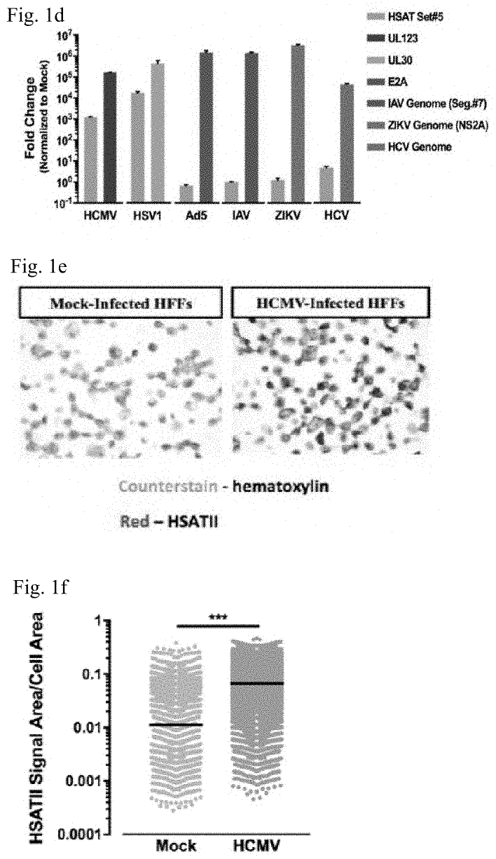

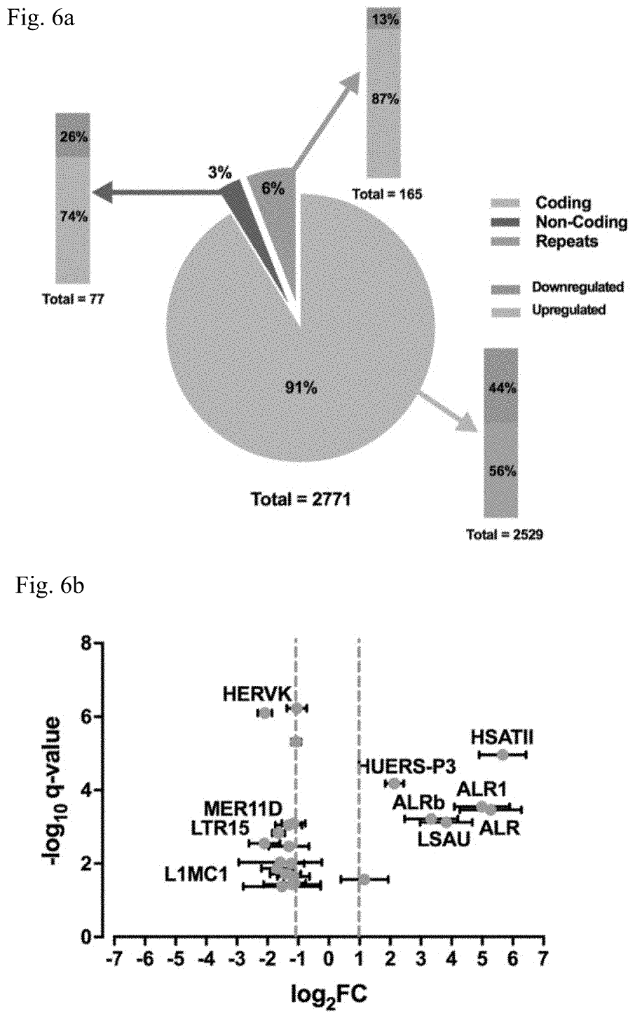

An assay of total RNA-seq was conducted to capture both coding and non-coding transcriptomes of acute HCMV infection in human foreskin fibroblasts (HFFs) (FIG. 6). With a focus on non-coding RNAs whose levels changed with infection, the inventors discovered that the majority of transcripts (74%) were downregulated at 48 hpi, and this tendency was the most profound for repetitive elements as 87% of them were decreased in HCMV-infected cells. Of the 13% of repeat elements upregulated upon infection, there was a striking (#100-fold) increase of HSATII RNA over that seen in mock-infected cells (FIG. 1a and FIG. 6). Importantly, the ability to induce HSATII expression was common for both the HCMV laboratory strain (AD169) and the more clinically relevant isolates (TB40/E and FIX) (FIG. 1a). The inventors tested HSATII expression in the same cell type infected with two other DNA viruses, herpes simplex virus (HSV1) and adenovirus (Ad5) to determine whether HSATII induction was indiscriminate cellular response to any infection. HSV1 increased HSATII transcript levels to an even greater extent (>1500-fold) but, surprisingly, Ad5 did not alter expression of the satellite RNA (FIG. 1a). By analyzing only uniquely mapped HSATII reads in the RNA-seq dataset, the inventors determined that HSATII in infected cells is produced preferentially from chromosome 1, 2, 10 and 16 and that HSATII accumulation from chromosome 16 was heavily favored following infection (FIG. 1b*—with the caveat that repeats often have high genomic diversity, abundant integration sites, and incomplete annotation). Of note, the inventors found that infected cells seem to have less diverse HSATII chromosomal expression patterns when compared to primary tumors. HSATII sequences were found to be often expressed in some cancers from the chromosome 7 locus26. The inventors determined that in tumors a higher percentage of HSATII transcripts also originated from chromosome 22 as well as other chromosomal loci (FIG. 1b). However, the preferential expression of HSATII in infected cells closely aligned with chromosomes where HSATII is a main constituent of the pericentromere 1 and which are largely responsible for the HSATII expression observed in cancer cells (FIG. 1b).

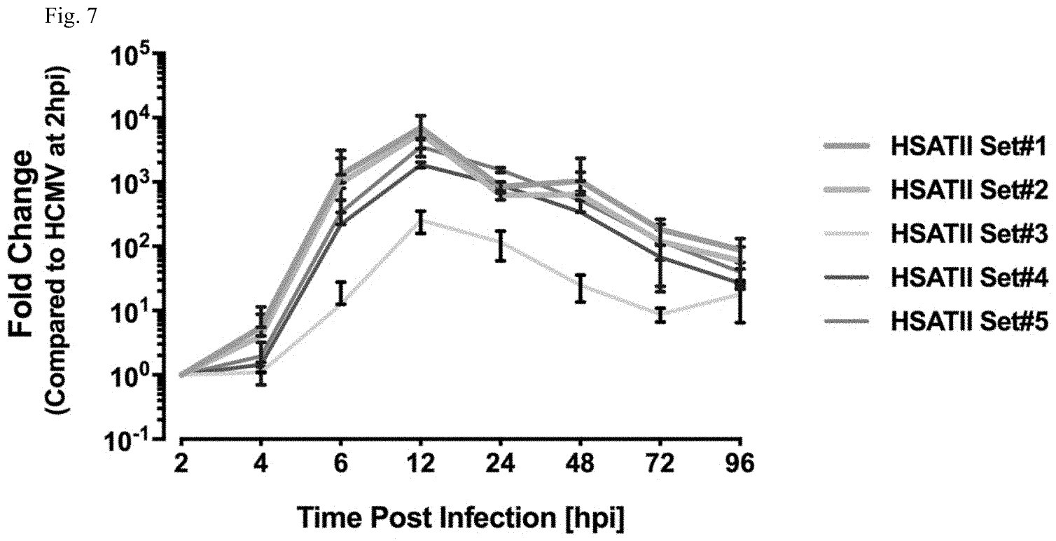

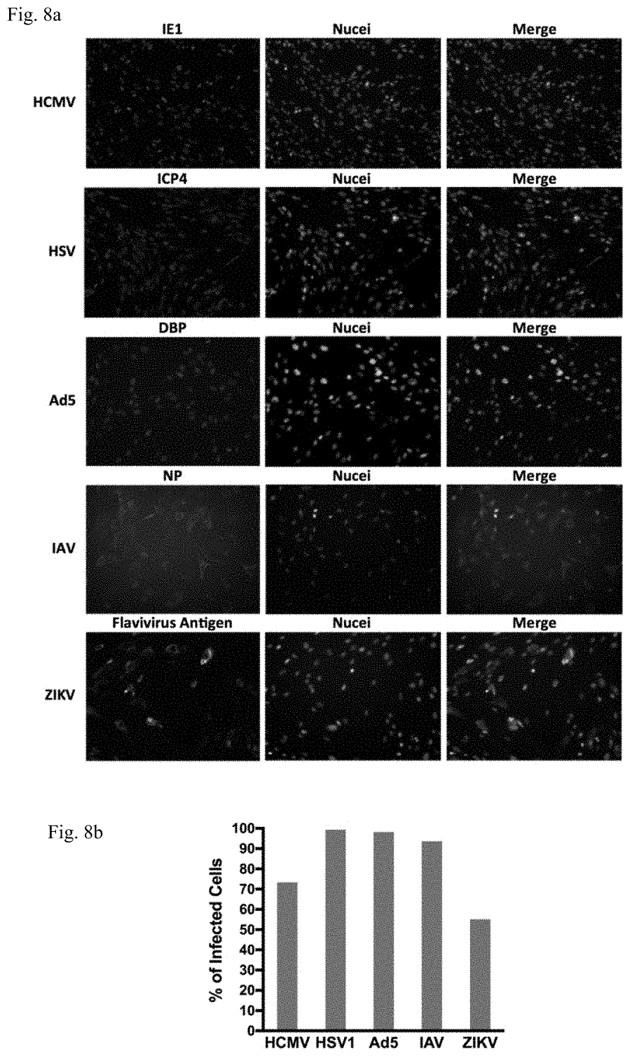

To validate the RNA-seq data, the inventors designed sets of HSATII-specific PCR primers (HSATII Se t #1-#5) based on highly expressed transcripts detected in HCMV-infected cells. Analysis of the kinetics of HSATII transcript accumulation in HCMV-infected fibroblasts demonstrated an initial induction during the immediate-early phase of infection at 6 hpi with continued increase up to the onset of viral DNA replication at 24 hpi (FIG. 1c). HSATII levels then decreased, but remained substantially elevated until the end of the viral replication cycle at 96 hpi. Interestingly, the kinetics of HSATII expression were cell type-specific. In HCMV-infected ARPE-19 epithelial cells, HSATII expression was accelerated and reached maximum at 12 hpi (FIG. 7). HSATII RNA was also induced in fibroblasts infected with HSV1; but Ad5, as well as several RNA viruses—influenza A (IAV), ZIKA virus (ZIKV) and hepatitis C virus (HCV)—failed to induce the probed HSATII sequences (FIG. 1d), even when close to 100% of cells were infected (FIG. 8).

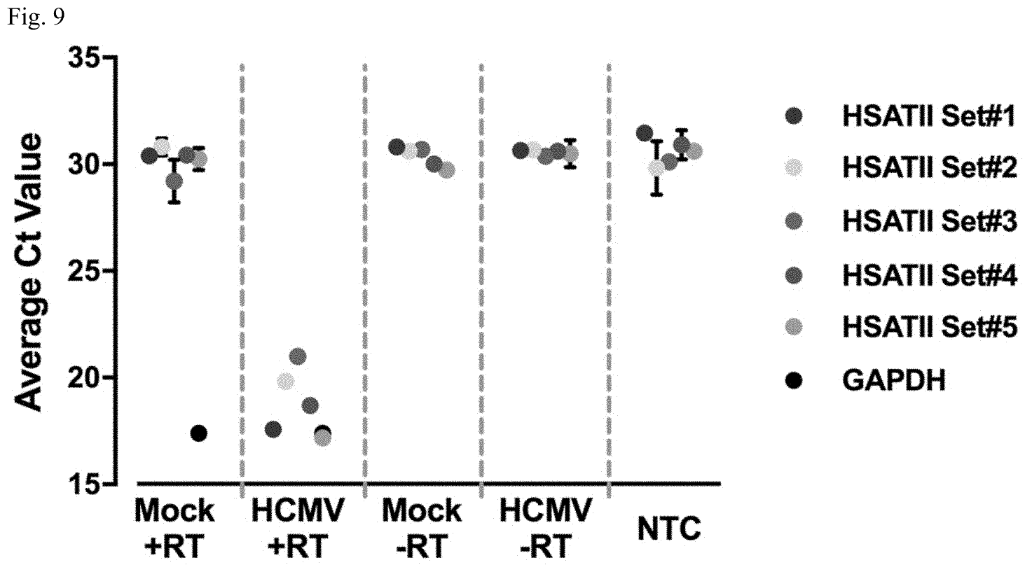

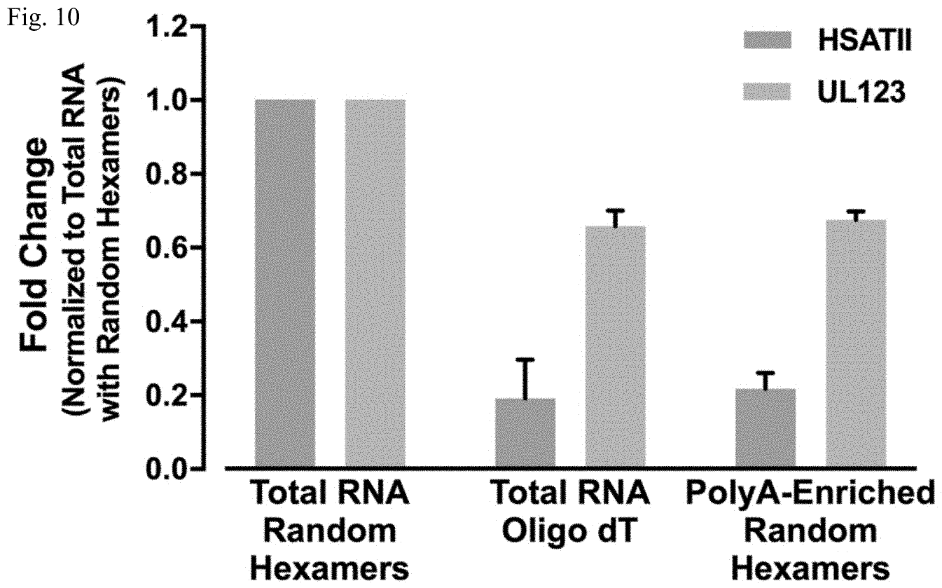

The detection of HSATII transcripts required a reverse transcription step before PCR amplification (FIG. 9), suggesting that HSATII transcripts in HCMV-infected cells do not create RNA-derived DNA intermediates, as observed in cancer cells. Perhaps the rapid HSATII induction or lack of reverse transcriptase activity in HCMV-infected cells, as opposed to malignant cells, may prevent the generation of DNA-containing intermediates. Moreover, in contrast to a control HCMV mRNA, UL123, HSATII RNA from infected cells was not retained on an oligo-dT matrix or efficiently amplified from oligo dT-based cDNA, indicating that it is predominantly not polyadenylated (FIG. 10). The lack of a polyA tail on HSATII transcripts was confirmed by inspecting unique HSATII reads. HSATII expression was also analyzed in mock- and HCMV-infected cells using an in situ hybridization (ISH) assay for detection of HSATII RNA. HCMV-infected cells showed a robust increase in a signal for HSATII RNA with the majority of signal localized in nuclei (FIG. 1e, f).

### Example 2—HCMV IE1 and IE2 Proteins Induce HSATII Expression

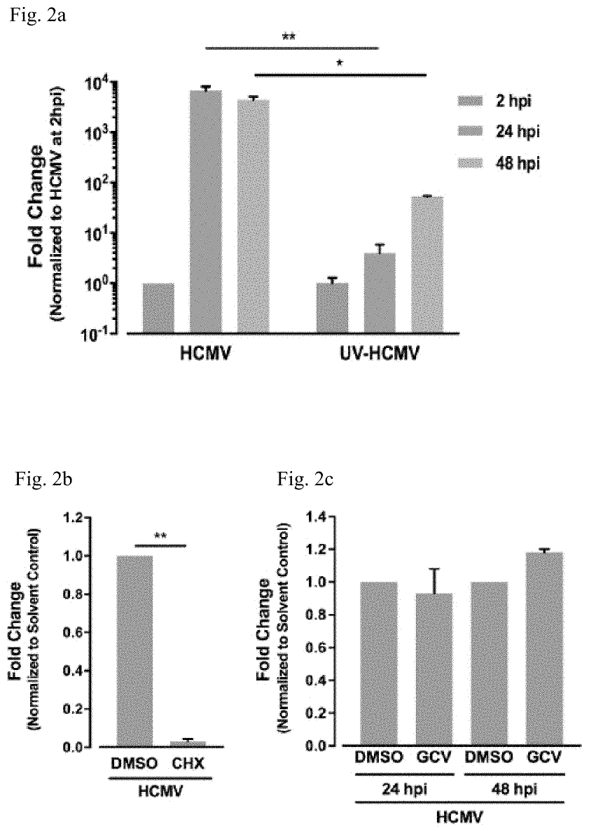

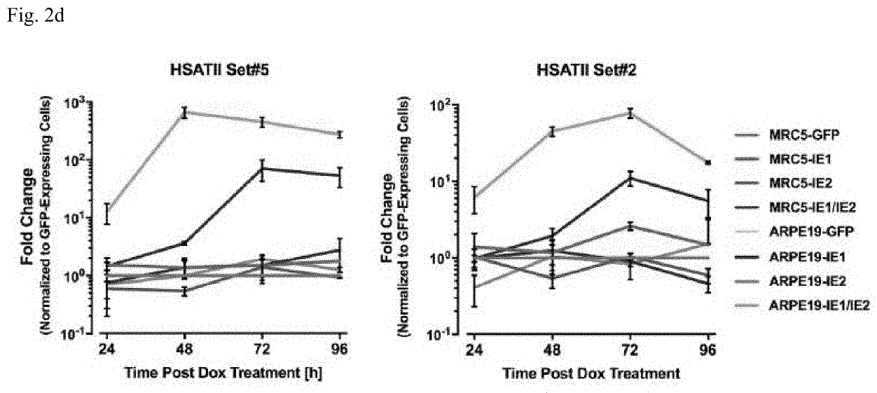

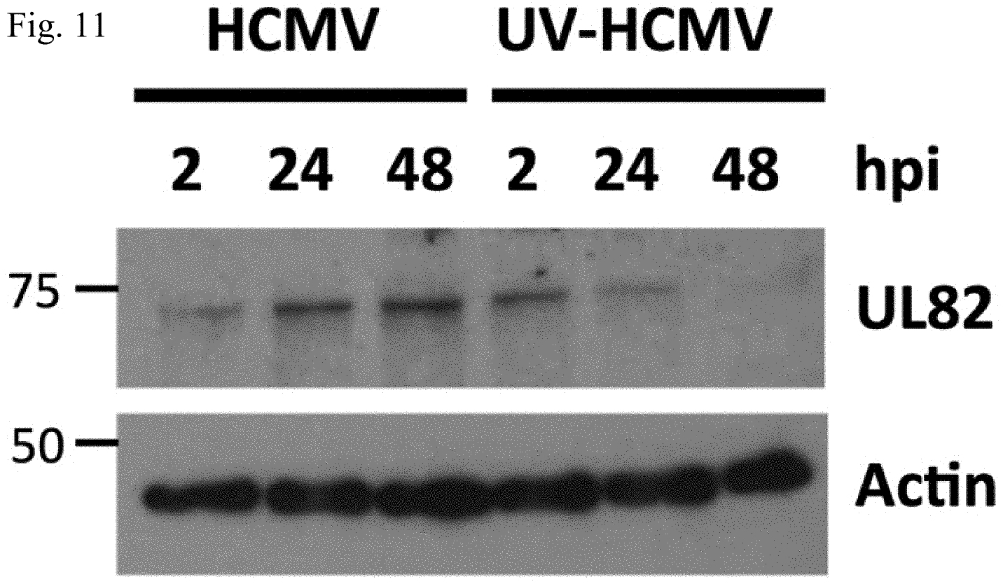

The inventors infected cells with replication-competent HCMV or replication-defective UV-irradiated virus. In comparison to cells receiving active virus, HSATII RNA induced by UV-irradiated virus was reduced by factors of ⁻1700 and ⁻100 at 24 and 48 hpi, respectively, as compared to its expression at 2 hpi (FIG. 2a). As a control, the inventors showed that the levels of a virion protein, pUL82 (pp71), increased following infection with replication-competent HCMV, but the tegument-delivered protein was degraded after infection with UV-irradiated virus with no new pUL82 accumulation (FIG. 11). These data reveal that active viral gene expression is necessary to induce HSATII expression. Cycloheximide (CHX) treatment strongly inhibited (33-fold reduction) HSATII accumulation compared to HCMV-infected cells treated with a solvent control (FIG. 2b), showing that de novo protein synthesis is needed to stimulate HSATII transcription. The viral DNA synthesis inhibitor, ganciclovir (GCV), which blocks the expression of late viral genes, did not change the HSATII levels at 24 hpi or 48 hpi (FIG. 2c), revealing that immediate early (IE) and/or early (E) viral protein expression was sufficient to induce HSATII accumulation. As a control, accumulation of RNA from the late UL99 gene was assayed at 48 hpi, and, as expected, it was blocked by the drug (FIG. 12).

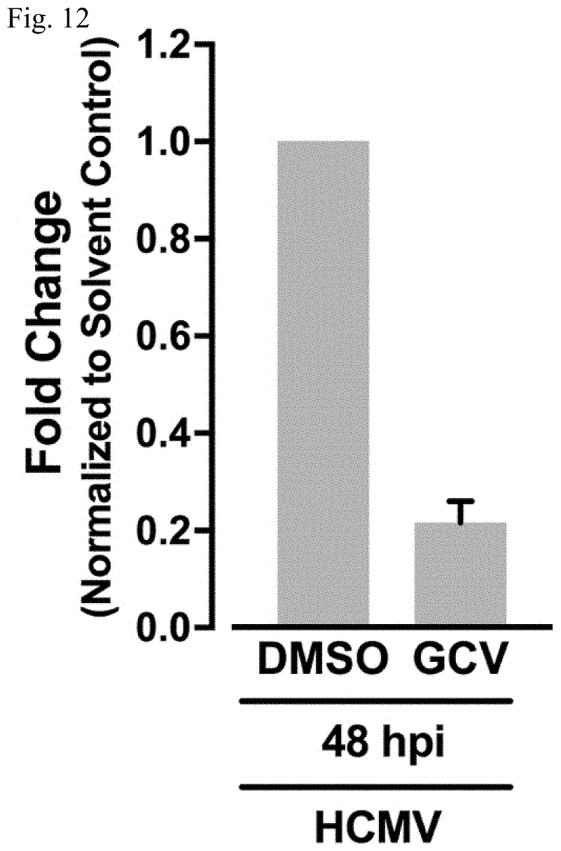

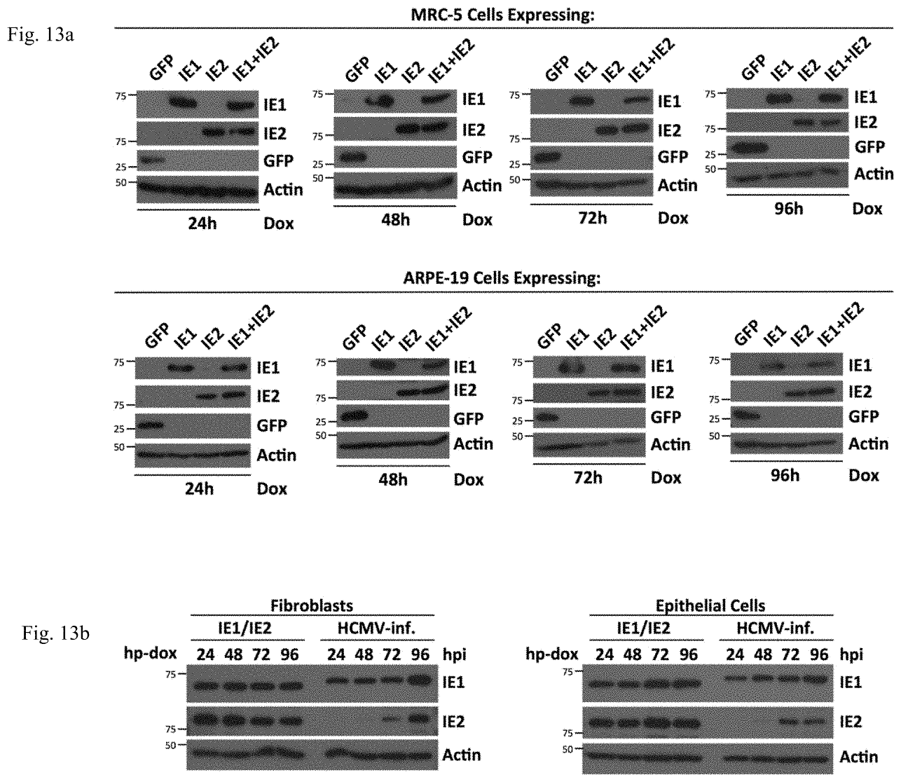

To identify which IE and/or E viral factor(s) were responsible for HSATII induction, the inventors tested the viral IE1 and IE2 proteins, which are known to be promiscuous transcriptional activators. MRCS fibroblasts and ARPE19 epithelial cells were prepared containing tetracycline-inducible IE1, IE2 or IE1+IE2 cDNAs, and Western blot assays confirmed induction of the viral proteins (FIG. 13). Although expression of IE1 or IE2 alone had little effect, expression of both proteins induced robust HSATII expression in fibroblasts and epithelial cells (FIG. 2d). The kinetics of HSAII expression was faster following induction of IE1+IE2-expression in epithelial cells than in fibroblasts, mimicking the difference evident in infected cells (FIG. 1c and FIG. 7). IE1 from protein lysates of IE1+IE2-expressing cells migrated faster than the protein from infected cells when subjected to electrophoresis in an SDS-polyacrylamide gel (FIG. 13), suggesting IE1 produced outside the context of infection might lack one or more modifications. This could reduce IE1 transactivation, since posttranslational modifications are known to affect the activity of IE1 and IE229-32. Further, the IE2 cDNA used to create IE2-inducible cells carries a single amino-acid substitution, A463T, which modestly reduces its transactivation activity compared to wild-type virus33. The inventors determined that IE1 and IE2 clearly act in concert to markedly induce the accumulation of HSATII transcripts from multiple chromosomal loci, as they are known to do for mRNA expression.

### Example 3—HSATII RNA Modulates HCMV RNA, Proteins and Progeny Levels

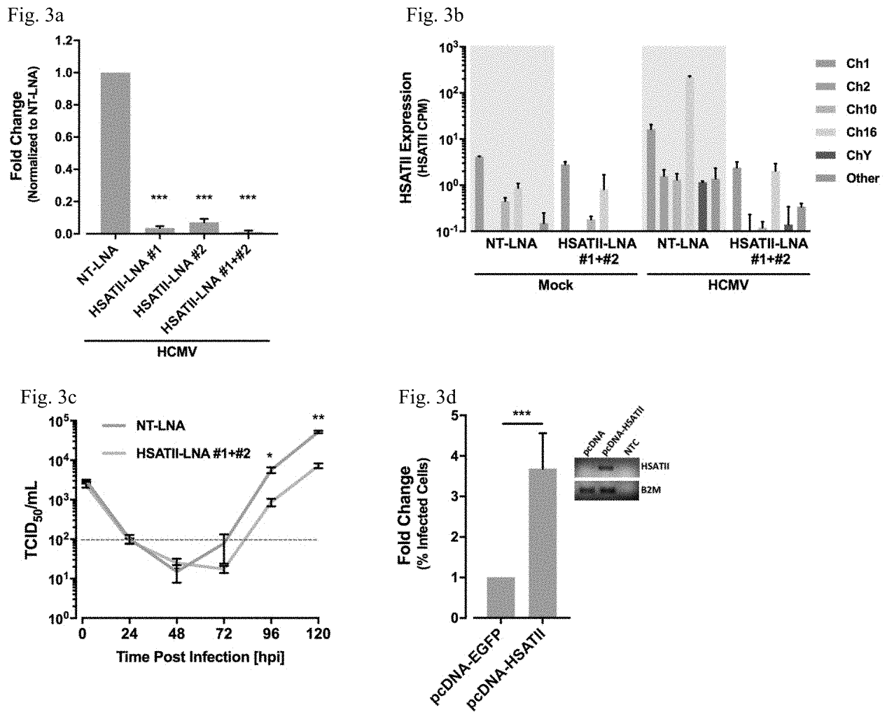

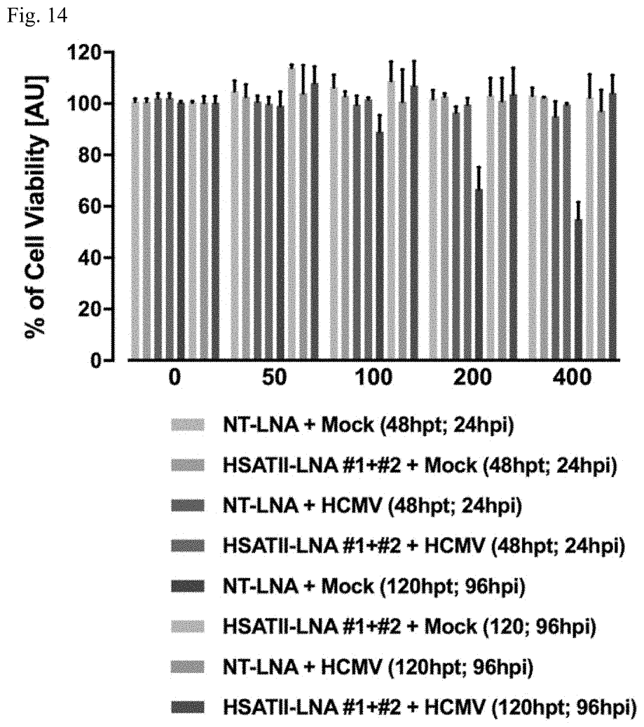

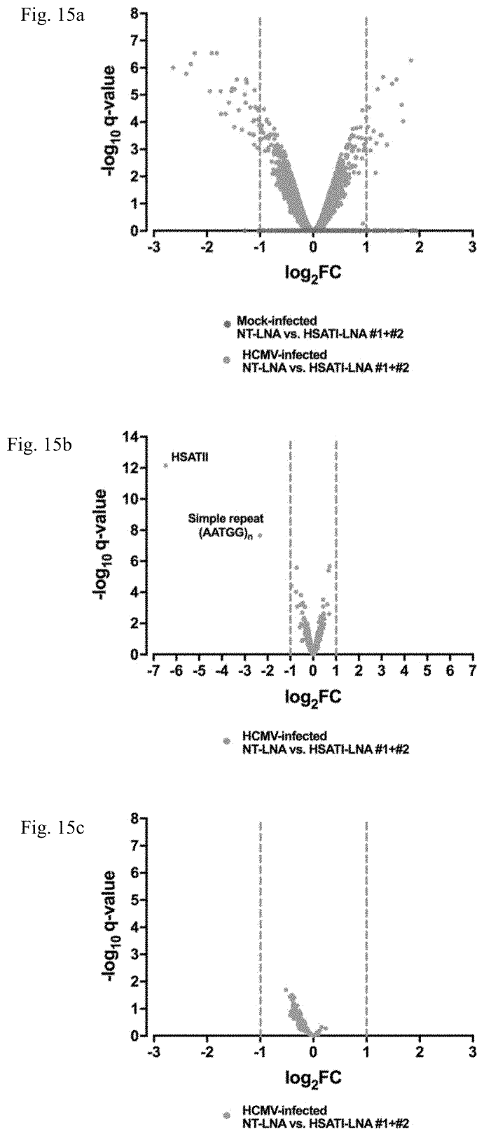

The inventors utilized locked nucleic acids (LNAs) that specifically target HSATII transcripts for degradation. The LNAs did not cause detectable nonspecific cellular toxicity (FIG. 14). Cells transfected with HSATII-specific LNAs (HSATII-LNAs) 24 h prior to infection had strongly decreased HSATII levels (FIG. 3a). RNA-seq analysis revealed that HSATII transcripts from all chromosomal loci in HCMV-infected cells were markedly decreased in HSATII-LNA-transfected cells compared with control NT-LNA transfected cells, but little effect on the low levels of HSATII RNAs was evident in mock-infected cells (FIG. 3b). Multiple cellular protein-coding transcripts were increased or decreased following LNA treatment, but no effect on coding RNA levels was evident in mock-infected cells (FIG. 15a). Additionally, HSATII-LNAs were very specific in downregulating HSATII versus other repeat RNAs (FIG. 15b). The HSATII RNAs, as a group, were reduced by a factor of 90, and only one simple repeat RNA [(AATGG)n] was reduced by a factor of five (FIG. 15b). However, the inventors detected only small number of simple repeat reads, which might result from self-priming in the PCR amplification step of the RNA-seq protocol. Further, the simple repeat reads might be related to expression of genes that have those repeats in their vicinity. Importantly, the (AATGG)n RNA was not induced by HCMV infection and its expression was not influenced by HSATII-LNAs in mock-infected cells.

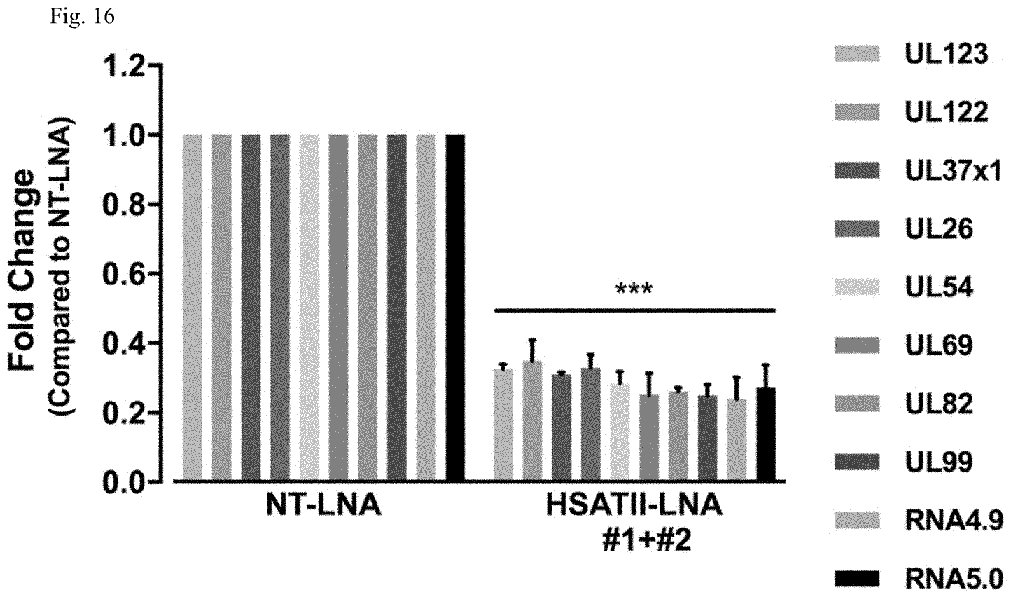

Tests were conducted of the effects of LNA-based HSATII knockdown on the production of extracellular HCMV progeny in fibroblasts. The ability of two individual HSATII-LNAs or their combination to efficiently decrease HSATII transcript levels (FIG. 3a) correlated with their effect on HCMV titer (FIG. 16). With the use of both HSATII-LNAs together, HSATII knockdown reduced the accumulation of infectious virus at 96 and 120 hpi by a factor of −8 as compared to controls when evaluated by TCID50 assay (FIG. 3c and FIG. 16). Ectopically overexpressed HSATII RNA (FIG. 3d, insert) had the opposite effect, increasing the infectious yield by a factor of −3.5× at 96 hpi (FIG. 3d). Together these data reveal that HSATII RNA participates in the production of HCMV progeny.

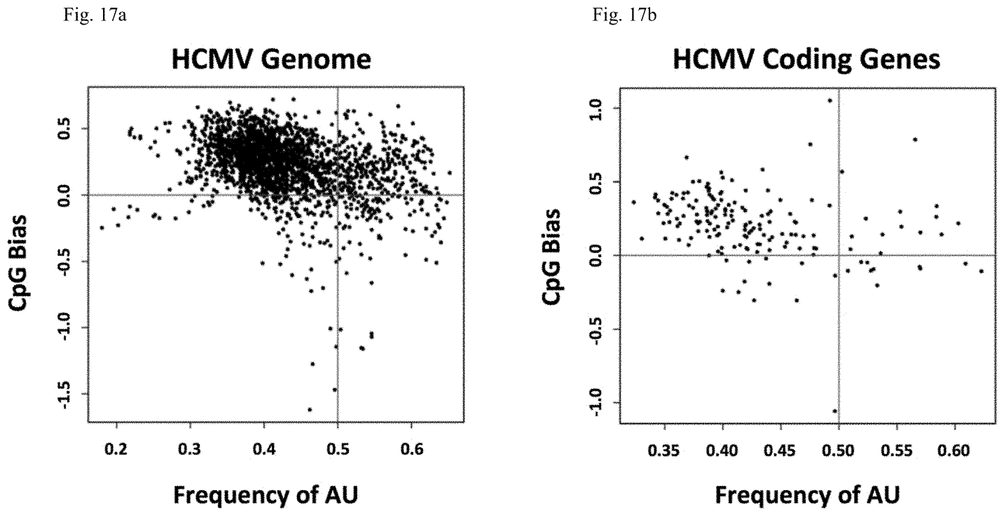

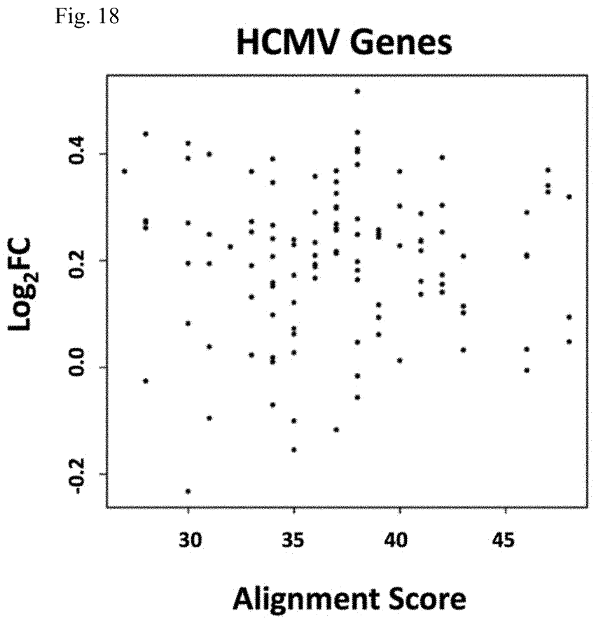

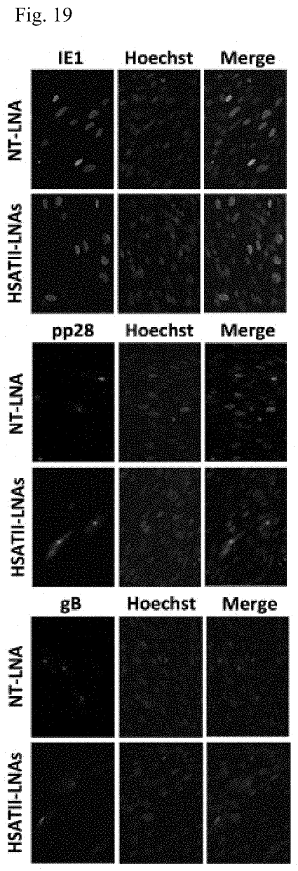

Tests were conducted on the effect of HSATII knockdown on levels of viral RNA, proteins, and genomic DNA (vDNA) in infected cells. For RNA analysis, the inventors quantified the expression of representatives from each of the three main classes of viral genes and HCMV long non-coding RNAs (lncRNAs). qRT-PCR determined HSATII suppression reduced levels of viral immediate-early (UL123, UL122, UL37xl), early (UL26, UL54), late (UL69, UL82, UL99) and lncRNAs (RNA4.9, RNA5.0 RNAs) at 96 hpi (FIG. 17). The reduction for each of the tested RNAs was on the order of 70%. HCMV has a higher GC-content (−57%) than the cell, and viral coding RNAs can have CpG motif overrepresentation. However, those CpG motifs are not in a background of AU-rich sequences—as it is the case for HSATII sequences, and are unlikely to react with HSATII-LNAs (FIG. 18). Furthermore, RNA-seq analysis did not detect any significant effect of HSATII-LNAs on differential expression of HCMV transcripts at 24 hpi as compared to their expression in NT-LNA-treated cells (FIG. 15c). Additionally, the inventors did not find any correlation between the differential expression of HCMV transcripts at 24 hpi in NT-LNA- and HSATII-LNA-treated fibroblasts and the sequence similarity of the corresponding HCMV transcript sequences and HSATII-LNAs (FIG. 19). This further reveals that there were no off-target effects of the HSATII-LNAs directed toward HCMV transcripts. In sum, the inventors discovered that the lower expression levels of of multiple HCMV transcripts HSATII-deficient cells arises from lower HSATII levels in those cells.

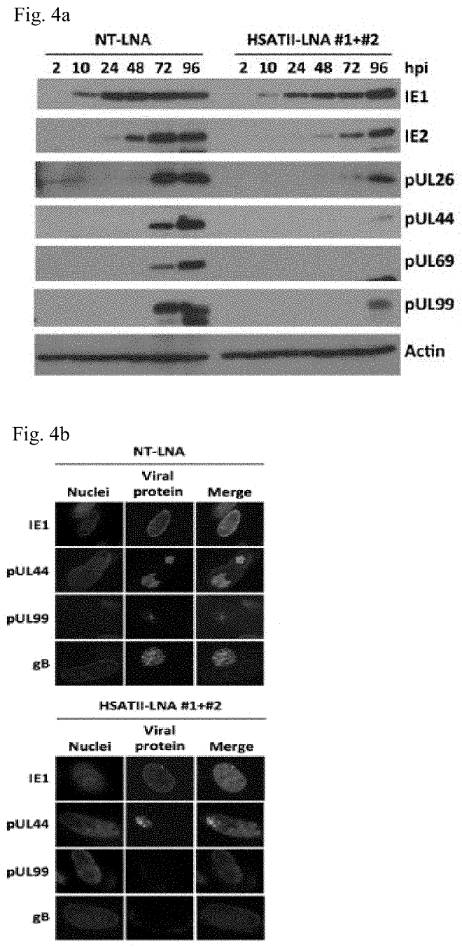

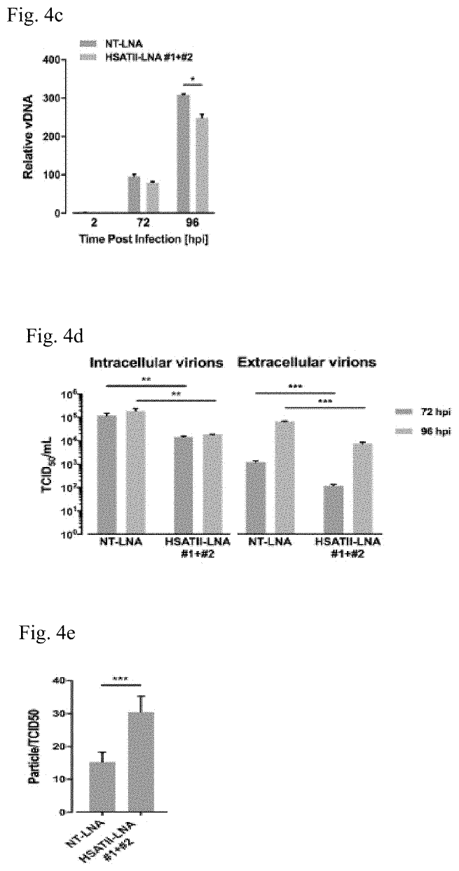

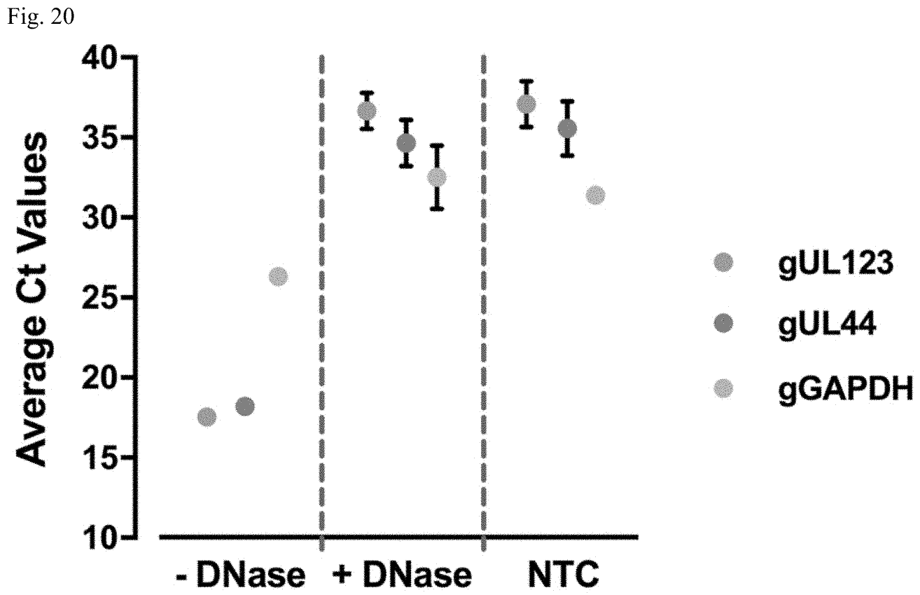

Western blot assays indicated that the level of IE1 protein was reduced by a factor of 2-3 at each time point examined between 10-72 hpi in HSATII knockdown cells, but it reached the same level as in cells where HSATII was expressed normally by 96 hpi (FIG. 4a). In contrast, IE2 and the early and late viral proteins accumulated to significantly lower levels at each time tested in HSATII-deficient cells. The IE1 protein, which is spread throughout the nucleus, and the pUL44 subunit of the viral DNA polymerase, which accumulates in viral replication centers, were localized normally in HSATII-deficient cells (FIG. 4b). However, the late pp28 and gB virion proteins, which normally accumulate in the cytoplasmic assembly compartment, were partially mislocalized in infected cells lacking HSATII. A portion of each virion protein was spread through the larger part of cytoplasm (FIG. 4b and FIG. 20). Thus, viral protein levels mimicked viral RNA levels, and portions of several late proteins were improperly localized. Consistent with perturbed viral protein expression and localization, HSATII knockdown reduced the level of intracellular vDNA to a limited extent (−20% reduction) at 96 hpi (FIG. 4c).

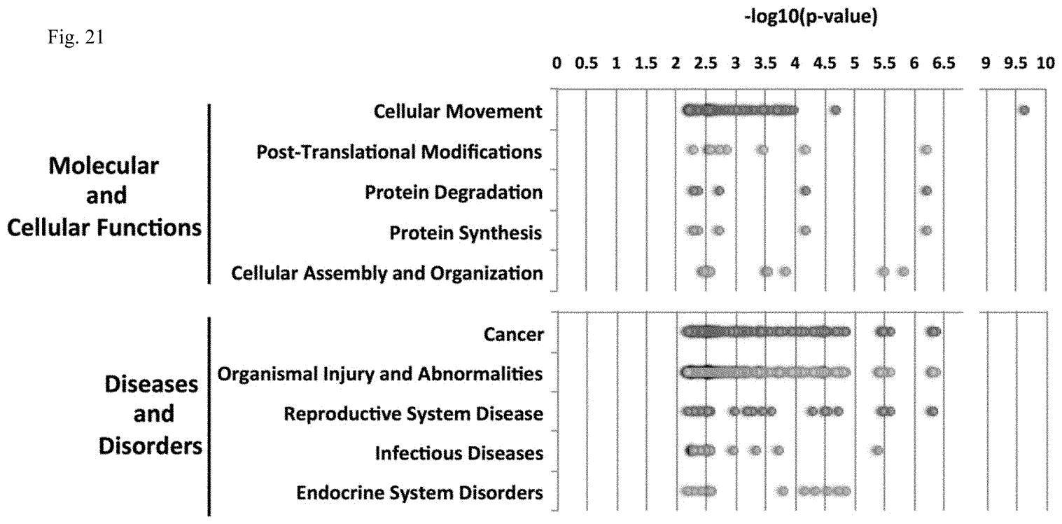

To further assess the effect of HSATII on virus production, monitoring was conducted of the accumulation of intracellular and extracellular virus at 72 and 96 hpi. When HSATII RNA was knocked down, infectious virus was reduced in both locations by a factor of −10 at both times after infection (FIG. 4d). As the viral titer represents not only the number of viral particles but also their infectivity, the particle/TCID50 ratio for extracellular viral particles was determined. By comparing DNase I-resistant vDNA to infectivity, the inventors discovered that virions released from HSATII-deficient cells are less infectious (−2-fold) than those from control cells (FIG. 4e). For the control, meanwhile, DNase treatment was effective in removing unprotected DNA (FIG. 21). Although intracellular DNA was reduced to a limited extent, the number and specific infectivity of virions was reduced in the absence of HSATII RNA, likely due to perturbations in the levels and localization of proteins that function during the late phase of infection.

### Example 4—HSATII RNA Alters Cellular RNA Levels and Cell Movement

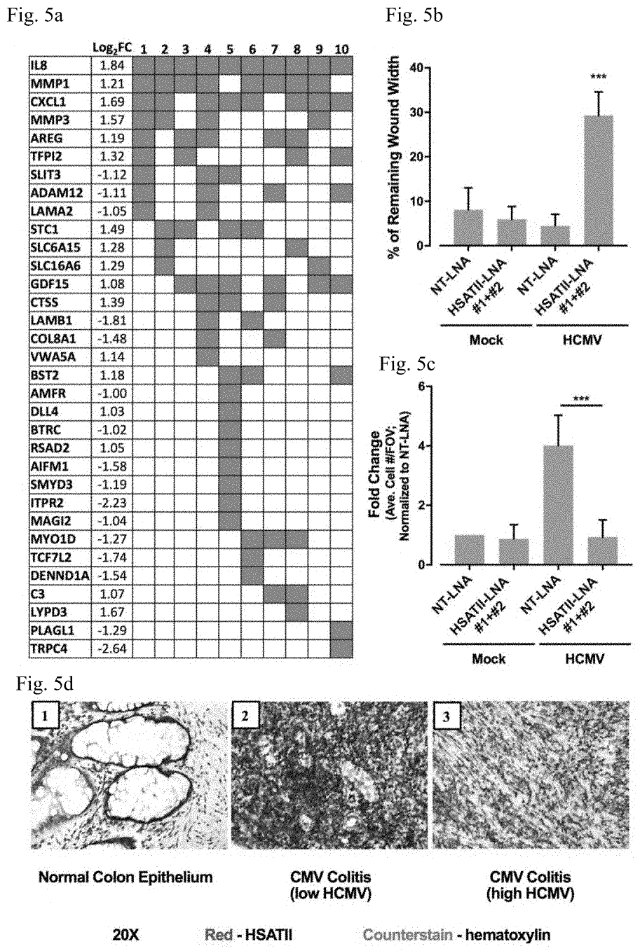

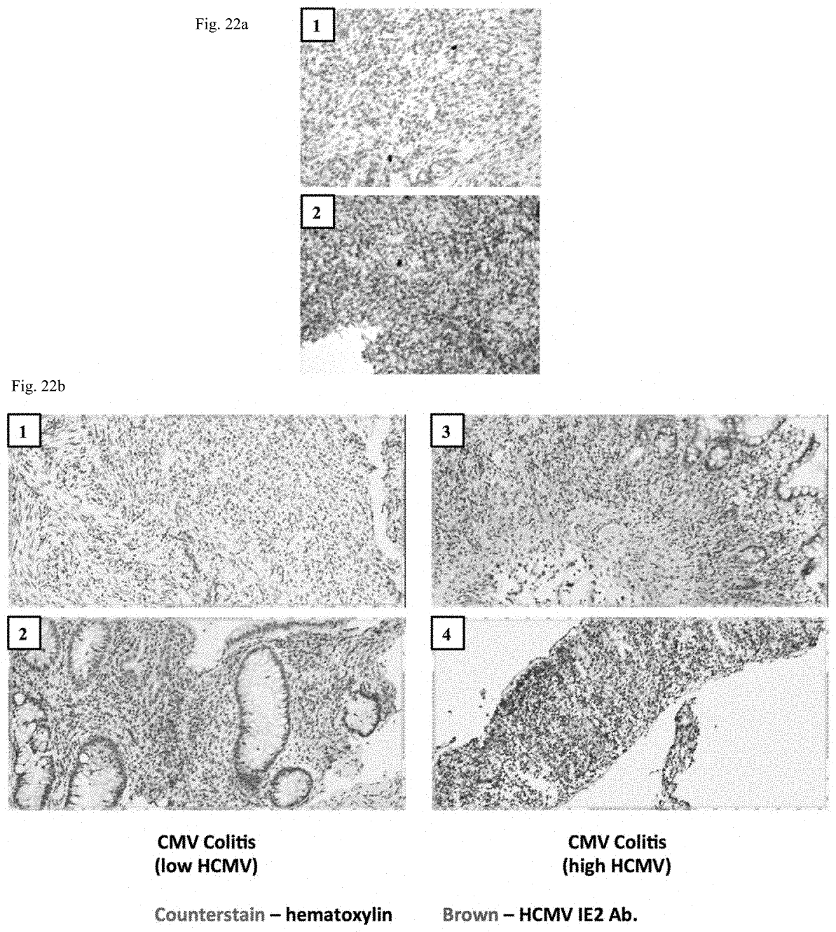

RNA-seq was used to monitor global gene expression of cells treated with control or HSATII-LNAs. No effect of LNA treatment was evident in mock-infected cells; in contrast, the levels of multiple cellular coding RNAs were modulated within infected cells (FIG. 15a). IPA and GSEA analyses of differentially regulated RNAs strongly associated virus-induced HSATII RNA with the regulation of protein stability and posttranslational modifications, and particularly with cellular movement (FIG. 5a and FIG. 22). Cells treated with HSATII-LNAs exhibited decreased expression of RNAs including ADAM12, TCF7L2, PLAGL1, SLIT3, DI02, and LPP, as well as increased levels of CXCL1, CXCL8, MMP1, MMP3, STC1 and CTSS (FIG. 5a). Of note, the latter genes are associated with inflammation and oncogenesis; thus, these tests further support the inventor's surprising discovery that HSATII RNA is involved in immune regulation and cancer progression for tumor cells.

The inventors have shown that reduced HSATII RNA levels in infected cells modulated expression of genes associated with cell movement. HCMV is known to modulate the motility of multiple cell types, a phenotype with potential to influence both HCMV spread and latency within its infected host. Since HCMV triggers high levels of HSATII RNA in epithelial cells (FIG. 1a and FIG. 7), a cell type playing an important role in HCMV pathogenesis and the wound healing process, the inventors examined the participation of HSATII RNA levels in wound closure or migration of infected epithelial cells. A wound-healing assay revealed that HCMV-infected cells lacking high HSATII levels were much slower in closing wounds compared to uninfected cells, and this effect was even more pronounced when compared to infected cells with normal, high levels of HSATII RNA (FIG. 5b). A transwell migration assay further demonstrated that cells characterized by a low HSATII RNA level were also less mobile (−4×) than HCMV-infected cells with a highly induced HSATII expression (FIG. 5c). Other data showed that transfection efficiency for exogenous expression of HSATII was too low in epithelial cells preventing assessment of results from the wound healing and transwell migration assays. These results show that HSATII induction promotes a transcriptional environment permissive for cell movement.

### Example 5—HSATII RNA is Elevated in CMV Colitis

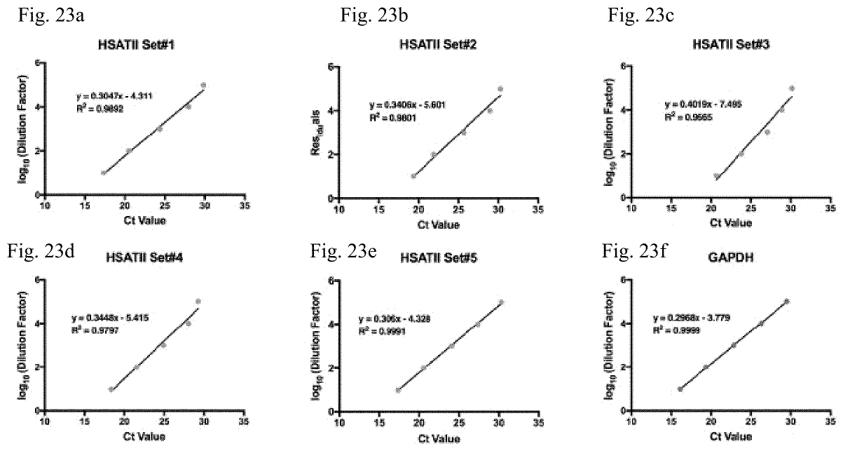

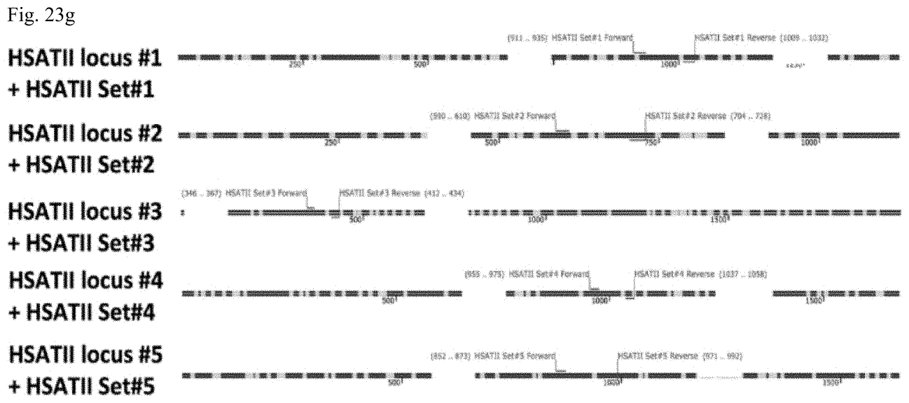

A hallmark of severe HCMV infection is the involvement of multiple organs. Infection of the gastrointestinal tract may lead to the onset of CMV colitis, which in rare cases of immunocompetent individuals resembles gastroenteritis and in patients with a compromised immune system is the second most frequent outcome of CMV disease after CMV retinitis. The inventors used RNA ISH to evaluate the levels of HSATII RNA in normal colon epithelium versus tissue biopsies from two patients manifesting low or high grade of CMV colitis. Low versus high grade was based on a standard immunohistochemical (IHC) assay staining IE and E CMV antigens (CCH2-UL44/DDG9-IE) or the IHC assay specifically staining HCMV IE2 protein (FIG. 5d and FIG. 23). The inventors found the latter staining method to have higher sensitivity (FIG. 5d and FIG. 23). IHC staining of colitis samples with the use of the inventors' IE2 antibodies is a novel approach and was utilized after determining that HCMV IE1 and IE2 proteins work cooperatively in inducing HSATII RNA (FIG. 2d). As with uninfected fibroblasts (FIG. 1e), normal colon epithelium was negative for HSATII-specific signal (FIG. 5d). The inventors found concordance between the level of CMV infection based on detection of viral proteins by IHC and the strength of the HSATII RNA signal (FIG. 5d and FIG. 23).

Identifying patients with CMV colitis is rare given the challenging diagnosis. The inventors determined that HCMV IE1 and IE2 proteins cooperate to induce HSATII expression (FIG. 2d), and the positive staining for IE2 protein in colitis samples is consistent with the possibility that elevated levels of HSATII could result from regulation by viral proteins in this tissue as well. Moreover, these results revealed that elevated HSATII RNA has a role in CMV colitis. This invention provides the first demonstration of elevated HSATII RNA in virally infected tissue.

There are numerous striking similarities between virus-infected cells and cancerous cells. These include, for example: manipulative interactions with the innate and adaptive immune system; metabolic changes and changes in cell division to provide substrates for virus and cellular replication; epigenetic alterations in cells to promote replication or spread of the virus or the cancer cell; and extensive communication between cells and tissues. Induction of HSATII RNA synthesis in virus-infected cells and many cancers appears to utilize all of these altered cellular processes for the benefit of the fitness of the cancer cells or the virus. HSATII RNA can affect the innate immune system inducing the synthesis of IL-6 and TNF-alpha. HSATII RNA and some viruses (i.e. avian Influenza A) share RNA nucleotide motifs that appear to be recognized by components of the innate immune system (such as ZAP) or pattern recognition receptors and this can result in evolutionary selection pressures that change the viral genome sequences with time and replication. The present invention, meanwhile, shows for the first time the role of HSATII in cellular motility, an important element in the virus and cancer cell fitness within a host.

### Example 6—HSATII LNAs Decreasing HSATII RNA Levels

RNA samples were collected at 24 hpi from HFFs transfected with NT-LNA or different HSATII-LNAs 24 h before HCMV (TB40/E-GFP) infection at 1 TCID50/cell. RT-qPCR was performed using HSATII-specific primers. GAPDH was used as an internal control. Data were averaged from at least three independent experiments and are presented as a fold change mean (SD). FIG. 24 demonstrates the effect of HSATII knockdown using four different HSATII-LNA on production of HCMV infectious particles from human foreskin fibroblasts (HFF). The data indicates that the combined HSATII-LNA #1 and HSATII-LNA #2 cause the most efficient HSATII knockdown. The HSATII knockdown caused by the combined HSATIILNA #1 and HSATII-LNA #2 is significantly more efficient than knockdown caused by HSATIILNA #1 or HSATII-LNA #2 alone. Unpaired, two-tailed t-test was used to measure significance. The asterisk represents p<0.05.

### Example 7—HSATII Role in HCMV Yield from Infected Fibroblasts

Media samples were collected at 96 hpi from HFFs transfected with NT-LNA or different HSATII-LNAs 24 h before HCMV (TB40/EGFP) infection at 1 TCID₅₀/cell. TCID₅₀/ml values were determined. Data were averaged from at least three independent experiments and are presented as a fold change mean (SD). FIG. 25 demonstrates the effect of HSATII knockdown using four different HSATII-LNA on production of HCMV infectious particles from human foreskin fibroblasts (HFF). The data indicates that the combined HSATII-LNA #1 and HSATII-LNA #2, which cause the most efficient HSATII knockdown (FIG. 1), also led to the most robust decrease of extracellular HCMV infectious particles. Cells treated with the combined HSATII-LNA #1 and HSATII-LNA #2 produced only ˜10% of extracellular HCMV viral particles produced by control cells treated with NT-LNA. Unpaired, two-tailed t-test was used to measure significance. One, two or three asterisks represent p<0.05, p<0.01, and p<0.001, respectively.

### Example 8—HSATII Role in HCMV Yield from Infected Epithelial Cells

Media samples were collected at 96 hpi from ARPE-19 cells transfected with NT-LNA or different HSATII-LNAs 24 h before HCMV (TB40-epi) infection at 1 TCID₅₀/cell. PFU/ml values were determined. Data were averaged from at least three independent experiments and are presented as a mean (SD). FIG. 26 demonstrates the effect of HSATII knockdown using four different HSATII-LNA on production of HCMV infectious particles from human retinal pigment epithelial cells (ARPE-19). The data indicates that the combined HSATII-LNA #1 and HSATII-LNA #2, which cause the most efficient HSATII knockdown (FIG. 1), also led to the most robust decrease of intracellular and extracellular HCMV infectious particles. Cells treated with the combined HSATII-LNA #1 and HSATII-LNA #2 produced only ˜0.6% of intracellular and ˜1% extracellular HCMV viral particles produced by control cells treated with NT-LNA. To compare, cells treated with HSATII-LNA #1 produced ˜33% of intracellular and ˜6% of extracellular HCMV viral particles produced by control cells treated with NT-LNA. Cells treated with HSATII-LNA #2 produced 23% of intracellular and 6% of extracellular HCMV viral particles produced by control cells treated with NT-LNA. Unpaired, two-tailed t-test was used to measure significance. One, two, three or four asterisks represent p<0.05, p<0.01, p<0.001 or p<0.0001, respectively.

### Example 9—Cells, Viruses, and Reagents

Human lung fibroblasts (MRC-5), human dermal fibroblasts (HDF; immortalized by expressing SV40 large T antigen) and human retinal pigment epithelial (ARPE-19) cells were from the American Type Culture Collection (ATCC). HCV-infected Huh7.5 cells are from Ploss lab (Princeton University). Primary human foreskin fibroblasts (HFF) and other fibroblasts were cultured in Dulbecco's Modified Eagles Medium (DMEM) supplemented with 10% fetal bovine serum (10% FBS/DMEM) (Sigma-Aldrich, St. Louis, Mo.). HFFs were used at passages 8-13. ARPE19 cells were cultured with added Ham's F-12 nutrient mixture (Sigma-Aldrich). 100 units ml-1 of penicillin (Sigma-Aldrich) and 95 μg ml-1 of streptomycin (Thermo Fisher Scientific, Waltham, Mass.) were added to media.

To construct IE1 and IE2 expressing cell lines, cDNAs encoding 72 kDa IE1 (IE-72) and 86 kDa IE2 (IE-86) from strain Towne were PCR amplified from pLXSN-IE169 and pLXSN-IE2, respectively. The IE2 cDNA contains missense mutations at methione 242 (M242I), eliminating the internal start responsible for generating the 40 kDa IE2-40 protein, and alanine 463 (A463T), which reduces IE2's transactivation activity by about 50%. A cDNA of monomeric EGFP was subcloned from a derivative of pEGFP-N3 (Clonetech) containing the mutation A2060. Tetracycline inducible cell lines expressing IE1, IE2, or EGFP were created by inserting each cDNA into pLVX-TetOne-Puro (Clonetech), producing VSV-G pseudotyped lentivirus particles in 293FT cells, concentrating lentivirus particles by ultracentrifugation over a 20% sorbitol cushion, and transducing MRC-5 or ARPE-19 cells. Stable cell lines were selected for 1 week in the presence of puromycin. Dual IE1 and IE2 expressing cells were created by cloning Towne IE2 into a derivative of pTetOne-Puro where the endogenous SV40-promoter-puromycin cassette was removed and a porcine teshovirus 2A-Neomycin geneblock (P2A-Neomycin) was inserted on the 3-prime-end of the reverse-Tetracycline transactivator (rtTA). Lentivirus particles were prepared as above. Stable lines were generated by co-transducing IE1 and IE2 lentivirus particles and selecting for 1 week in the presence of puromycin and G418.

Two GFP-tagged viruses derived from clinical isolates, TB40/E-GFP, FIX-GFP, as well as a GFP-tagged laboratory strain AD169-GFP were used in these studies. TB40-epi designates TB40/E virus produced by growing the TB40/E strain grown in ARPE-19 cells. Viruses were produced from BAC clones transfected with pp71 expression plasmid into HFFs, MRC-5 or ARPE-19 cells to generate viral progeny of wild-type growth characteristics. Viruses were purified by centrifugation through a sorbitol cushion (20% sorbitol, 50 mM Tris-HCl.1 mM MgCl2, pH 7.2), concentrated and resuspended in DMEM. Viral titers were determined using a tissue culture infectious dose 50 (TC1D50) assay on HFFs or ARPE-19 cells, and infections were performed at a multiplicity of 3 TC1D50/cell or as designated. UV-inactivation of TB40/E-GFP virions was performed by 4 sequential UV irradiations of viral inoculum using Auto Cross Link settings (UV Stratalinker 2400; San Diego, Calif.).

HSV-1 strain F were grown in Vero cells. Pooling cell-associated virus, obtained by sonication, with cell-free virus, produced viral stocks. HSV-1 titers were determined using TC1D50 assay. Fibroblasts were infected with HSV1 at a multiplicity 3 TCID50/cell. Adenovirus (Ad5) was kindly provided by S. J. Flint (Princeton University). Ad5 titer was determined on MRC-5 cells by a focus forming assay and is expressed as focus forming units (FFU). Fibroblasts were infected with Ad5 at a multiplicity 10 FFU/cell. Influenza A virus [IAV; A/PR/8/1934(H1N1) (ATCC)] titer was determined using TCID50 assay. HFFs were infected with IAV at a multiplicity 3 TCID₅₀/cell in Flu infection buffer E %*&* containing FCHK BSA_{B} G AgD7l L-1-tosylamido-2-phenylethyl chloromethyl ketone (TPCK)-treated trypsin (Thermo Fisher Scientific) and 0.1% FBS]. Zika virus (ZIKV; ZIKV/1947/UG/MR766) titer was determined using a plaque assay. HDFs were infected with ZIKV at a multiplicity 10 PFU/cell. Hepatitis C Virus (HCV; JCI strain expressing Cre recombinase) titer was determined on Huh-7.5 cells using TCID₅₀ assay. Huh-7.5 cells were infected with HCV at a multiplicity 1 TCID₅₀/cell.

Following a 2-h absorption period for all viruses, inoculum was removed, cells were washed twice with complete medium and collected at indicated time points post infection. When indicated, experimental HCMV viral titers were also determined by assaying for IE1-positive cells on reporter plates.

To measure the portion of cells within a culture that were infected, fibroblasts were fixed with methanol and stained using mouse antibodies anti-HCMV IE1 (1B12), anti-HSV ICP4 (hybridoma supernatant), anti-Ad5 E2, anti-IAV nucleoprotein (HB-65), or anti-Flavivirus Group Antigen Antibody (Sigma) and goat anti-mouse Alexa Fluor-488 conjugated secondary antibody (Invitrogen). Nuclei were counterstained with Hoechst 33342. Cells were visualized and the percentage of viral antigen-positive cells was calculated from at least 20 fields of view using the Operetta high-content imaging and analysis system (PerkinElmer).

Cyclohexamide (Sigma-Aldrich) and ganciclovir (Sigma-Aldrich) were dissolved in DMSO and used at 100 μg ml⁻¹ or 50 1.1M concentrations, respectively. Doxycycline (Sigma-Aldrich) was dissolved in water and used at 2 μg ml⁻¹. Puromycin was dissolved in water and used at 1.5 1.1 g ml⁻¹ (MRC-5) or 21.1 g ml⁻¹ (ARPE-19). G418 was dissolved in water and used at 800 μg ml-1 (MRC-5) or 1 mg ml-1 G418 (ARPE-19).

### Example 10—RNA Analysis

For RNA sequencing (RNA-Seq) analysis, RNA from HCMV−, HSV1-, or Ad5-infected cells at defined multiplicities of infectious units/cell and appropriate mock-infected cells was collected in QIAzol Lysis Reagent (Qiagen) at 48, 9 or 24 hpi, respectively. The specific times of sample collection were chosen to capture the viral replication cycles at their halfway points. RNA was isolated using the miRNeasy Mini Kit (Qiagen). DNA was removed from samples using Turbo DNase (Thermo Fisher Scientific) and RNA quality was analyzed using the Bioanalyzer 2100 (Agilent Technologies, Santa Clara, Calif.). cDNA sequencing libraries were prepared by the Penn State College of Medicine Genome Sciences Facility using the TruSeq Stranded Total RNA with Ribo-Zero kit (Illumina, San Diego, Calif.) for rRNA depletion, and subjected to multiplexed sequencing (RNA-Seq) using Rapid HiSeq2500 sequencer (Illumina) for 100 cycles in paired-end, rapid mode (2×100 bp).

RNA-Seq data was de-multiplexed based on indexes and raw RNA reads were quality filtered as follows. First, ends of the reads were trimmed to remove N's and bases with quality less than 20. After that, the quality scores of the remaining bases were sorted and the quality at the 20th percentile was computed. If the quality at the 20th percentile was less than 15, the whole read was discarded. Also, reads shorted than 40 bases after trimming were discarded. If at least one of the reads in the pair failed the quality check and had to be discarded, we discarded the mate as well. Human, HCMV, HSV1 and Ad5 fasta and annotation (.gtf) files were created for mapping by combining sequences and annotations from Ensembl annotation, build 37, repbase elements (release 19) and TB40/E (EF999921.1), FIX (GU179289), AD169 (FJ5275630), HSV1 (GU734771), or Ad5 (AC000008) when appropriate. To that created concatenated human-virus genomes, quality filtered reads were mapped using STAR aligner.

Aligned reads were assigned to genes using the featureCounts function of Rsubread package with the external Ensembl annotations. This produced the raw read counts for each gene. Gene expression in terms of log 2-CPM (counts per million reads) was computed and normalized across samples using the trimmed mean of M-values method (TMM), as implemented in the calcNormFactors function of edgeR package. Differential expression analysis was performed using limma package. Expression data were used in conjunction with the weights computed by the voom transformation.

To calculate the percent of HSATII reads originating from each chromosome in infected cells and in selected samples from the Cancer Genome Atlas (TCGA), the inventors identified uniquely mapped reads that exclusively overlapped with HSATII repeat. The number of normalized counts of HSATII reads mapped to each chromosome was computed. Next, the percentage of these reads mapping to each chromosome was calculated by dividing their number by the total number of HSATII reads and multiplying by 100%. The inventors only considered samples with at least 100 HSATII reads. TCGA samples were comprised of 12 LUAD (Lung Adenocarcinoma), 10 COAD (Colon Adenocarcinoma), 5 BRCA (Breast Invasive Carcinoma), 4 KIRC (Kidney Renal Clear Cell Carcinoma), 4 UCEC (Uterine Corpus Endometrial Carcinoma), and 3 BLCA (Bladder Urothelial Carcinoma) tumors.

CpG bias of the viral genes and contiguous 500 bp segments of the viral genome was computed using statistical methods developed by Greenbaum et al.

The best local alignment of LNAs to each of the viral genes was identified using water program of EMBOSS package with gap opening and gap extension penalties set to 10 and default score matrix. The best alignment score for each gene was plotted against loge (fold change of gene expression) between HCMV-infected cells treated with NT-LNA or HSATII-LNA #1+#2. The maximal score was chosen out of the score for LNA #1 and LNA #2.

Ingenuity Pathway Analysis (IPA) cloud software (Qiagen) was used to overlay differentially expressed genes onto global molecular network information incorporated in the Ingenuity Pathway Knowledge Base. The Core Analysis in IPA was used to organize the data sets into gene ontologies and to identify predicted biological functions and processes relevant to the data set based on t value determining the probability of association with a given gene set.

Gene Set Enrichment Analysis (GSEA) was also used to investigate the data set overlap with annotated gene sets comprising the Molecular Signature Database (MSigDB). A matrix of differentially expressed genes from the data set significantly matching identified MSigDB gene sets was composed and ordered based on a number of overlapping genes, t value determining the probability of association with a given gene set and a false discovery rate q-value.

For quantitative reverse transcription PCR (qRT-PCR) analysis, cells were collected in QIAzol Lysis Reagent (Qiagen). To fractionate RNA, DNA and proteins chloroform was added; samples were spun at 12,000×g for 15 min. at 4° C. RNA from an aqueous layer was isolated using the miRNeasy Mini kit (QIAGEN) according to the manufacturer's instructions. RNA samples were stored at −80° C. DNA contaminants were removed from the samples using the TURBO® DNase Kit (Invitrogen by Thermo Fisher Scientific) according to the manufacturer's instructions. cDNA was made using random hexamers (Invitrogen by Thermo Fisher Scientific) and Superscript III Reverse Transcriptase Kit (Invitrogen by Thermo Fisher Scientific) according to the manufacturer's instructions. Quantitative PCR (qPCR) was performed using SYBR Green master mix (Applied Biosystems by Thermo Fisher Scientific, Foster City, Calif.) on the QuantStudio 6 Flex-Real Time PCR System (Applied Biosystems by Thermo Fisher Scientific). For a semiquantitative PCR, product amplification was carried out using PTC-225 thermocycler (MJ Research Inc., BioRad Laboratories), with the following PCR mix: 10×PCR Reaction Buffer with MgCl2 (Roche), 1.25 units of Taq DNA Polymerase (Roche) and a 200 !M concentration of each deoxynucleotide (Thermo Fisher Scientific). The performance of HSATII specific primer sets was tested for uniformity and consistency across serially diluted cDNA sample and show a high level of linearity during amplification (FIG. 23 a-e).

Primer sequences used in qRT-PCR reactions are listed in Table 1. Transcript levels were analyzed using the AACt method and GAPDH or B2M were used as an internal control. Data were averaged from at least three experiments and are presented as a fold change mean (SD). Student's t-test were performed and t value was used to measure a statistical significance between samples.

TABLE 1
Primer Sequences used in qPCR.

| Target Sequence | Forward Primer (5′ 3′) | Reverse Primer (5′ 3′) |
|---|---|---|
| HSV1 | CATCACCGACCCGGAGAGGG | GGGCCAGGCGCTTGTTGGTG |
| UL3O | AC | TA |
| Ad5 E2A | GTGTAGACACTTAAGCTCGCC | CTTCAAACTACTGCCTGACC |
| | TT | AAGT |
| IAV | CCACTGAAGTGGCATTTGGC | CTGTAGTGCTGGCTAAAACC |
| Genome | | |
| ZIKV | CCGCTGCCCAACACAAG | CCACTAACGTTCTTTTGCAGAC |
| Genome | | AT |
| HCV Genome | GTCTAGCCATGGCGTTAGTA | CTCCCGGGGCACTCGCAAGC |
| HSATII | CCAATGGAATCAGAAATAACC | TCCTTTCATTTCCATTCAATG |
| Set#1 | ATCA | AGG |
| HSATII | TGTGATCATCATCGAACGGAC | ATGAGTCCTTCCTTTTCAATT |
| Set#2 | | TCAT |
| HSATII | TCGTGTCTATTCAAAGGTTCC | ACGAGTGGAATCGATAGCC |
| Set#3 | A | ATAA |
| HSATII | GATTCCACTTGAGTCCGTTAG | GGAATCATCGTCGAATGGAG |
| Set#4 | | |
| HSATII | TTGGTGATTCCACTGGATTTCT | TCGGATGGAATCAATGAAG |
| Set#5 | | GGA |
| HCMV | TGCTGTGCTGCTATGTCTTAG | TTGGTTATCAGAGGCCGCTT |
| UL123 | AGG | GG |
| HCMV | TGACCGAGGATTGCAACG | CGGCATGATTGACAGCCTG |
| UL122 | | |
| HCMV | TCCCGCCTTGGTTAAGA | ACTGGGCGTTGTTGAGCATA |
| UL37x1 | | |
| HCMV | CCAGCAGCTTCCAGTATTC | ACCTGGATCTGCCCTATC |
| UL26 | | |
| HCMV | TGCTTTCGTCGGTGCTCTCTAA | TGTGCGGCAGGTTAGATTGA |
| UL54 | G | CG |
| HCMV | ACGAGTGTCAGAACGAGATGT | TGAAACGATAGGGTGCCAA |
| UL69 | GC | CGC |
| HCMV | AGACGTCGAAGCGGTAACAA | AGTCGTCAAGGCTCGCAAAG |
| ULE12 | CG | AC |
| HCMV UL99 | ACGACAACATCCCTCCGACTTC | TCTGTTGCCGCTCCTCGTTATC |
| HMCV RNA4.9 | TTGACAAGCGATGGAGGACC | TGAGCGGTTGTGTTGGATGA |
| CMV RNA5.0 | ACACCGTCAGGGAACACATC | GTGTATCGAGCCACCGTGAT |
| HSATII- | CCGCCAGTGTGCTGGAATTC | GCCGCCAGTGTGATGGATATC |
| pcDNA | | A |
| GAPDH | CAAGAGCACAAGAAGAAGAGAG | CTACATGGCAACTGTGAGGAG |
| B2M | GCCCAAGATAGTTAAGTGGGATCG | TCCAAATGCGGCATCTTCAAACC |

### Example 11—Protein Analysis

Cells were either harvested using protein lysis buffer [50 mM Tris-HCl at pH 7.5 (Thermo Fisher Scientific), 5 mM ethylenediaminetetraacetic acid (EDTA; Thermo Fisher Scientific), 100 mM sodium chloride (Thermo Fisher Scientific), 1% Triton X-100 (Thermo Fisher Scientific), 0.1% sodium dodecyl sulfate (SDS; Roche), and 10% glycerol (Sigma)] or Trizol. If Trizol was used, upon RNA/DNA/protein fractionation and the removal of RNA and DNA fractions, proteins were precipitated by adding 2-propanol. After pelleting proteins at 12000× g for 10 min at 4° C., the pellet was washed with of 0.3 M GuHCl/95% EtOH, washed with 100% EtOH, resuspended in 1:1 1% sodium dodecyl sulfate (SDS):8M Urea/1M tris(hydroxymethyl) aminomethane (Tris) and sonicated. Protein samples were stored at −80° C. Protein samples were mixed with 6×SDS sample buffer (325 mM Tris pH 6.8, 6% SDS, 48% glycerol, 0.03% bromophenol blue) containing 9% 2-mercaptoethanol (Sigma). Proteins were separated by electrophoresis (SDS-PAGE) and transferred to ImmunoBlot polyvinylidene difluoride (PVDF) membranes (BioRad Laboratories). Western blot analyses were performed using mouse monoclonal antibodies anti-IE1 (1B12; 1:500 dilution), anti-IE2 (3A9; 1:500 dilution), anti-pUL26 (7H1-5; 1:100 dilution), pUL44 (CMV ICP36; 1:80,000 dilution; Virusys; Taneytown, Md.; cat. #CA006), anti-pUL69 (10E11; 1:100 dilution), anti-pUL82 (10G11; 1:100 dilution), anti-pUL99 (10B4-29; 1:100 dilution, anti-GFP (1:1400 dilution; Sigma; cat. #11814460001) and anti-a-actin-HRP (1:100,000 dilution; Abcam; cat. #ab49900). Goat anti-mouse antibody (1:10,000 dilution; Jackson ImmunoResearch Laboratoriesm Inc.; cat. #115-035-003) conjugated with horseradish peroxidase was used as secondary antibodies. Western blots were developed using WesternSure ECL Detection Reagents (Licor).

### Example 12—DNA Analysis

Cells were harvested and DNA was isolated using the DNA Blood & Tissue Kit (Qiagen). Intracellular viral DNA was quantified from total intracellular DNA. Extracellular viral DNA was isolated from sample media collected at 96 hpi. Media was treated with 30 units of DNase I (Invitrogen by Thermo Fisher Scientific, Carlsbad, Calif.) according to the manufacturer's recommendations. Virions in the media were lysed and isolated using the DNA Mini Kit (QIAGEN, Hilden, Germany) according to the manufacturer's instructions.

vDNA and cellular DNA copy numbers were determined based on standard curves of viral genomic UL44 (Forward: 5′-GTGCGCGCCCGATTTCAATATG-3′, Reverse: 5′-GCTTTCGCGCACAATGTCTTGG-3′ or cellular genomic GAPDH (Forward: 5′-CCCCACACACATGCACTTACC-3′, Reverse: 5′-CCTAGTCCCAGGGCTTTGATT-3′) amplified from serially diluted HCMV TB40-BAC4 DNA or pUC18-gGAPDH DNA, respectively. Data were averaged from at least three experiments and are presented as a fold change mean (SD). Student's t-test were performed and t value was used to measure a statistical significance between samples.

### Example 13—HSATII RNA Knockdown

Locked nucleic acid oligonucleotides were designed to target identified, highly abundant HSATII transcripts from different chromosomal loci. The most effective LNAs: HSATII-LNA #1 (5′-CCATTCGATAATTCCG-3′), HSATII-LNA #2 (5′-GATTCCATTCGATGAT-3′), or a mixture of both (HSATII-LNAs (#1+#2) were used for experiments as indicated. Lipofectamine RNAi Reagent (Thermo Fisher Scientific, Waltham, Mass.) and LNAs were resuspended in Opti-MEM medium (Thermo Fisher Scientific) according to the manufacturer's instructions. The final LNA concentration applied to cells was 100-200 nM. Non-target scrambled sequence LNA (NT-LNA; 5′-AACACGTCTATACGC-3′) was used as a negative control. HFFs and ARPE-19 cells were incubated for 24 h before being mock- or HCMV-infected. Cells were collected at the indicated time post infection using QIAzol buffer (QIAGEN, Hilden, Germany) and stored at −80° C. until sample processing.

To measure potential toxicity, HFFs were treated with LNA at concentrations ranging from 0 to 400 nM for 24 h prior HCMV infection at a multiplicity of 1 TCID₅₀/cell or were mock infected. At indicated time points, the Cell Titer 96 AQueous One Solution Cell Proliferation Assay (Promega, Madison, Wis.) was performed according to the manufacturer's instructions. Absorbance was measured at 490 nm using the SpectraMax Plus 384 Microplate reader (Molecular Devices, Sunnyvale, Calif.). Data is presented as % viable cells and were averaged from at least three experiments and are presented as mean (SD).

### Example 14—Plasmid Transfection

HFFs at 70% confluency were transfected with 1 μg of pcDNA3.1 (Addgene) or pcDNA-HSATII (a generous gift of Arnold Levine) using X-tremeGENE 9 DNA Transfection Reagent (Roche) according to the manufacturer's instructions. 24 h later, plasmid-transfected cells were infected with TB40/E-GFP at a multiplicity of 3 TCID50/cell. Media and RNA samples were collected at 96 hpi and stored at −80° C.

### Example 15—Cell Migration Assays

To perform wound healing assays, confluent monolayers of NT-LNA- or HSATII-LNA-transfected ARPE-19 cells were infected with TB40-epi at a multiplicity of 3 TCID50/cell or were mock infected. At 2 hpi, cells were washed to remove inoculum and scratching the cell monolayer with 1-mL pipet created wounds. The process of wound closure was monitored in time and pictures of wounds were taken using the Nikon Eclipse TE2000-U inverted microscope. The average wound width (in arbitrary units) of ARPE-19 cells was calculated from 6 measurements for each experimental arm from the captured images using ImageJ software. Results are plotted as a mean percent of remaining wound width (SD).

To perform transwell migration assay, NT-LNA- or HSATII-LNA-transfected ARPE-19 cells were infected with TB40-epi at a multiplicity of 3 TCID50/cell or mock-infected. At 6 hpi, cells were trypsinized and 5×10⁴ cells were seeded onto each filter in FBS-free medium containing ITS Liquid Media Supplement (Sigma-Aldrich). After 24 h at 37° C./5% CO2, filters were washed with 1×PBS and fixed in methanol. Non-migrated cells were removed with a cotton swab, and nuclei of migrated cells on the bottom surface of the filter were stained with Hoechst 33342 and were imaged by the Nikon Eclipse TE2000-U inverted microscope. Migrated cell number was quantified from 6 measurements for each experimental arm from the captured images using ImageJ software. Results are plotted as a fold change mean (SD) of average cell number per field of view (FOV).

### Example 16—RNA In Situ Hybridization (ISH) Assay

To analyze HSATII levels in HCMV-infected cells, HFFs were infected with HCMV at a multiplicity of 1 TCID₅₀/cell or mock-infected. At 24 hpi, cells were collected, washed with 1×PBS and resuspended in human plasma (Sigma-Aldrich). To facilitate sample coagulation, 13 NIH units of thrombin (Sigma-Aldrich) were added to each sample. Cells were then fixed in 10% formaldehyde for 4 h. The fixed pellets were transferred to biopsy cassettes. Automated ISH assays for HSATII RNA was performed using the ViewRNA eZ-L Detection Kit (Affymetrix by Thermo Fisher Scientific) on the BOND RX IHC and ISH Staining System with BDZ 6.0 software (Leica Biosystems Inc., Buffalo Grove, Ill.). Cell pellets were formalin-fixed and paraffin-embedded +FFPE) and cut in 5-μm sections on slides and processed automatically from deparaffinization, through ISH staining and hematoxylin counterstaining. Automatic coverslipper was used for coverslipping slides. Briefly, slides were baked for 1 h at 60° C., and placed on the BOND RX for processing. The BOND RX user-selectable settings were the ViewRNA ez-L Detection 1-plex (Red) protocol and ViewRNA Dewax1; ViewRNA HIER2 (90) 5 min; ViewRNA Enzyme 2 (5 min); ViewRNA Probe Hybridization 3 h. With these settings, the RNA unmasking conditions for the tissue consisted of a 5-minute incubation at 90° C. in Bond Epitope Retrieval Solution 2 (Leica Biosystems) followed by 5-minute incubation with Proteinase K from the BOND Enzyme Pretreatment Kit at 1:1000 dilution (Leica Biosystems). The HSATII (Affymetrix; Cat #VA1-10946) RNA-targeting Probe was diluted 1:40 in ViewRNA Probe Diluent (Affymetrix) for use on the automated platform. Diluted Probe Set, diluted Proteinase K, and ViewRNA eZ-L Detection Kit were loaded onto BOND RX prior to starting the run. After the run, post rinsing with water and drying for 30 min. at room temperature, slides were dipped in xylene, and mounted using HistoMount solution (Life Technologies by Thermo Fisher Scientific). HSATII signal from ISH experiments was quantified based on the ratio of HSATII signal area to cell area using BDZ 6.0 software.

To analyze HSATII levels in human biopsies of HCMV colitis, normal colon and two CMV positive colitis biopsies were analyzed. It is of note that identifying these patients is complicated and rare given the difficulty in the diagnosis of CMV colitis. Both patients had ulcerative colitis on immunosuppressive medications predisposing them to CMV infection. The diagnosis was made with biopsy of the colon and immunohistochemistry analysis performed by a board-certified anatomic pathologist. Immunohistochemical expression of the CMV was evaluated by deparaffinizing FFPE sections by baking them for 1 hour at 60° C. IHC staining was done on the BondRx using the BOND Polymer Refine Detection kit (Catalogue No. DS9800). Antigen retrieval was carried out with citrate buffer at pH 6 for 10 mins using Bond Epitope Retrieval Solution 1 (Leica Biosystems). Mouse monoclonal antibodies against HCMV (antibody mixture to infected cell lysate, clone CCH2+DDG9, Sigma-Aldrich); HCMV IE2 (clone 3H9) were diluted in Bond Primery Antibody Diluent (Leica Biosystems Inc.) and signal was detected by the Polymer Refine Kit (Leica Biosystems Inc.) and protocol F on a Leica Bond Rx Autostainer. Automated ISH assay for HSATII RNA was performed as described for HCMV-infected fibroblasts.

1. A composition comprising:
a polynucleotide that inhibits HSATII expression, activity, or function and a pharmaceutically acceptable carrier suitable for injection, wherein the polynucleotide comprises an siRNA molecule, shRNA molecule, or a locked nucleic acid molecule.

2. The composition according to claim 1, wherein the polynucleotide is a siRNA molecule.

3. The composition according to claim 1, wherein the polynucleotide is a shRNA molecule.

4. The composition according to claim 1, wherein the polynucleotide is a locked nucleic acid molecule.

5. The composition according to claim 1, wherein the sequence of the polynucleotide consists essentially of either (5′-CATTCGATAATTCCG-3′) or (5′-GATTCCATTCGATGAT-3′) or a conservative variant thereof.

6. The composition according to claim 1, wherein the composition consists essentially of a combination of two polynucleotides, one with a sequence that consists essentially of (5′-CATTCGATAATTCCG-3′) and the other with a sequence that consists essentially of (5′-GATTCCATTCGATGAT-3′) or conservative variants thereof.

7. The composition of claim 1, wherein the pharmaceutically acceptable carrier is selected from the group consisting of an emulsion, liposome, microspheres, immune stimulating complex, nanospheres, montanide, squalene, cyclic dinucleotides, complementary immune modulators, and combinations thereof.

8. A method of treating a subject comprising:
administering to a subject that has cancer, a viral infection, or a tumor the composition of claim 5 under conditions effective to treat the subject for the disease or disorder.

9. The method of claim 8, wherein the polynucleotide sequence consists essentially of either (5′-CATTCGATAATTCCG-3′) or (5′-GATTCCATTCGATGAT-3′) or a conservative variant thereof.

10. The method of claim 9, wherein the polynucleotide is a locked nucleic acid.

11. The method of claim 8, wherein the composition comprises a combination of two polynucleotides, one with a sequence that consists essentially of (5′-CATTCGATAATTCCG-3′) and the other with a sequence that consists essentially of (5′-GATTCCATTCGATGAT-3′) or conservative variants thereof.

12. The method of claim 8, wherein each polynucleotide is a locked nucleic acid.

13. A method of treating a subject comprising:
administering to a subject afflicted with cancer the composition of claim 5 under conditions effective to treat the subject.

14. The method of claim 13, wherein the polynucleotide sequence consists essentially of either (5′-CATTCGATAATTCCG-3′) or (5′-GATTCCATTCGATGAT-3′) or a conservative variant thereof.

15. The method of claim 14, wherein the wherein the polynucleotide is a locked nucleic acid.

16. The method of claim 13, wherein the composition comprises a combination of two polynucleotides, one with a sequence that consists essentially of (5′-CATTCGATAATTCCG-3′) and the other with a sequence that consists essentially of (5′-GATTCCATTCGATGAT-3′) or conservative variants thereof.

17. The method of claim 16, wherein each polynucleotide is a locked nucleic acid.

18. A method of treating a subject comprising:
administering to a subject that has cancer, a viral infection, or a tumor the composition of claim 6 under conditions effective to treat the subject for the disease or disorder.

19. A method of treating a subject comprising:
administering to a subject afflicted with cancer the composition of claim 6 under conditions effective to treat the subject.


SEQUENCE LISTING (50 sequences)

SEQ ID NO: 1 - DNA, 22 bp, Homo sapiens
catcaccgacccggagagggac

SEQ ID NO: 2 - DNA, 22 bp, Homo sapiens
gggccaggcgcttgttggtgta

SEQ ID NO: 3 - DNA, 23 bp, Homo sapiens
gtgtagacacttaagctcgcctt

SEQ ID NO: 4 - DNA, 24 bp, Homo sapiens
cttcaaactactgcctgaccaagt

SEQ ID NO: 5 - DNA, 20 bp, Homo sapiens
ccactgaagtggcatttggc

SEQ ID NO: 6 - DNA, 20 bp, Homo sapiens
ctgtagtgctggctaaaacc

SEQ ID NO: 7 - DNA, 17 bp, Homo sapiens
ccgctgcccaacacaag

SEQ ID NO: 8 - DNA, 24 bp, Homo sapiens
ccactaacgttcttttgcagacat

SEQ ID NO: 9 - DNA, 20 bp, Homo sapiens
gtctagccatggcgttagta

SEQ ID NO: 10 - DNA, 20 bp, Homo sapiens
ctcccggggcactcgcaagc

SEQ ID NO: 11 - DNA, 25 bp, Homo sapiens
ccaatggaatcagaaataaccatca

SEQ ID NO: 12 - DNA, 24 bp, Homo sapiens
tcctttcatttccattcaatgagg

SEQ ID NO: 13 - DNA, 21 bp, Homo sapiens
tgtgatcatcatcgaacggac

SEQ ID NO: 14 - DNA, 25 bp, Homo sapiens
atgagtccttccttttcaatttcat

SEQ ID NO: 15 - DNA, 22 bp, Homo sapiens
tcgtgtctattcaaaggttcca

SEQ ID NO: 16 - DNA, 23 bp, Homo sapiens
acgagtggaatcgatagccataa

SEQ ID NO: 17 - DNA, 21 bp, Homo sapiens
gattccacttgagtccgttag

SEQ ID NO: 18 - DNA, 20 bp, Homo sapiens
ggaatcatcgtcgaatggag

SEQ ID NO: 19 - DNA, 22 bp, Homo sapiens
ttggtgattccactggatttct

SEQ ID NO: 20 - DNA, 22 bp, Homo sapiens
tcggatggaatcaatgaaggga

SEQ ID NO: 21 - DNA, 24 bp, Homo sapiens
tgctgtgctgctatgtcttagagg

SEQ ID NO: 22 - DNA, 22 bp, Homo sapiens
ttggttatcagaggccgcttgg

SEQ ID NO: 23 - DNA, 18 bp, Homo sapiens
tgaccgaggattgcaacg

SEQ ID NO: 24 - DNA, 19 bp, Homo sapiens
cggcatgattgacagcctg

SEQ ID NO: 25 - DNA, 17 bp, Homo sapiens
tcccgccttggttaaga

SEQ ID NO: 26 - DNA, 20 bp, Homo sapiens
actgggcgttgttgagcata

SEQ ID NO: 27 - DNA, 19 bp, Homo sapiens
ccagcagcttccagtattc

SEQ ID NO: 28 - DNA, 18 bp, Homo sapiens
acctggatctgccctatc

SEQ ID NO: 29 - DNA, 23 bp, Homo sapiens
tgctttcgtcggtgctctctaag

SEQ ID NO: 30 - DNA, 22 bp, Homo sapiens
tgtgcggcaggttagattgacg

SEQ ID NO: 31 - DNA, 23 bp, Homo sapiens
acgagtgtcagaacgagatgtgc

SEQ ID NO: 32 - DNA, 22 bp, Homo sapiens
tgaaacgatagggtgccaacgc

SEQ ID NO: 33 - DNA, 22 bp, Homo sapiens
agacgtcgaagcggtaacaacg

SEQ ID NO: 34 - DNA, 22 bp, Homo sapiens
agtcgtcaaggctcgcaaagac

SEQ ID NO: 35 - DNA, 22 bp, Homo sapiens
acgacaacatccctccgacttc

SEQ ID NO: 36 - DNA, 22 bp, Homo sapiens
tctgttgccgctcctcgttatc

SEQ ID NO: 37 - DNA, 20 bp, Homo sapiens
ttgacaagcgatggaggacc

SEQ ID NO: 38 - DNA, 20 bp, Homo sapiens
tgagcggttgtgttggatga

SEQ ID NO: 39 - DNA, 20 bp, Homo sapiens
acaccgtcagggaacacatc

SEQ ID NO: 40 - DNA, 20 bp, Homo sapiens
gtgtatcgagccaccgtgat

SEQ ID NO: 41 - DNA, 20 bp, Homo sapiens
ccgccagtgtgctggaattc

SEQ ID NO: 42 - DNA, 22 bp, Homo sapiens
gccgccagtgtgatggatatca

SEQ ID NO: 43 - DNA, 22 bp, Homo sapiens
caagagcacaagaagaagagag

SEQ ID NO: 44 - DNA, 21 bp, Homo sapiens
ctacatggcaactgtgaggag

SEQ ID NO: 45 - DNA, 24 bp, Homo sapiens
gcccaagatagttaagtgggatcg

SEQ ID NO: 46 - DNA, 23 bp, Homo sapiens
tccaaatgcggcatcttcaaacc

SEQ ID NO: 47 - DNA, 15 bp, Homo sapiens
cattcgataattccg

SEQ ID NO: 48 - DNA, 16 bp, Homo sapiens
gattccattcgatgat

SEQ ID NO: 49 - DNA, 15 bp, Homo sapiens
cattcgataattccg

SEQ ID NO: 50 - DNA, 16 bp, Homo sapiens
gattccattcgatgat